(19) United States
(12) Patent Application Publication
(10) Pub. No.: US 2026/0315561 A1
(43) Pub. Date: Oct. 15, 2026

(54) BOTANICAL COMPOSITIONS AND METHODS FOR THE MODULATION OF THE GUT MICROBIOME

(71) Applicant: CAN TECHNOLOGIES, INC., Wayzata, MN (US)

(72) Inventors: Rachel Balder, Forest Lake, MN (US); Anirikh Chakrabarti, Mechelen (BE); Briana Kristen Kozlowicz, Hopkins, MN (US); Adam Jeffrey Rogers, North Liberty, IA (US); Maria Isabel Sardi, Plymouth, MN (US)

(73) Assignee: CAN TECHNOLOGIES, INC., Wayzata, MN (US)

(21) Appl. No.: 19/485,830

(22) PCT Filed: May 22, 2024
(86) PCT No.: PCT/US2024/030471

§ 371 Date: Nov. 19, 2025

Related U.S. Application Data
(60) Provisional application No. 63/503,990, filed on May 24, 2023.

(51) Int. Cl. A61K 36/258; A23K 10/30; A23K 50/40; A61K 36/07; A61K 36/21; A61K 36/235; A61K 36/24; A61K 36/288; A61K 36/33; A61K 36/48; A61K 36/53; A61K 36/605; A61K 36/62; A61K 36/638; A61K 36/67; A61K 36/74; A61K 36/889; A61P 1/00

(52) U.S. Cl. CPC A61K 36/258; A23K 10/30; A23K 50/40; A61K 36/07; A61K 36/21; A61K 36/235; A61K 36/24; A61K 36/288; A61K 36/33; A61K 36/48; A61K 36/53; A61K 36/605; A61K 36/62; A61K 36/638; A61K 36/67; A61K 36/74; A61K 36/889; A61P 1/00

(57) ABSTRACT

The present disclosure provides a botanical composition including a botanical extract, where the botanical extract is present in the botanical composition in an amount effective to produce a beneficial effect on a gut health of an animal. The beneficial effect can include one or more effect on the gut health of an animal that can include one or more improvements in gut health and immune health of an animal. The botanical extract can include an extract of dandelion leaf, dandelion leaf, ground dandelion leaf, dandelion root, Japanese ginseng, asafoetida, privit, common fennel, Indian mulberry, Rangoon creeper, long pepper, Indian lotus, cayenne/chili pepper, velvet foot winter mushroom, sugar apple, lion's mane mushroom, coriander, saw palmetto, Spanish dagger, mound lily yucca, carob, field lupine, goat's head, sweet prickly pear, partridge berry, bugleweed, Stelmatrocryption khasianum, Amaranthus spinosus, and elderberry, or any combinations thereof. Other aspects are also provided herein.

### CROSS-REFERENCE TO RELATED APPLICATIONS

This application claims the benefit of U.S. Provisional Application No. 63/503,990, filed May 24, 2023, which is incorporated by reference herein in its entirety.

### TECHNICAL FIELD

The present disclosure relates to botanical compositions for the modulation of the gut microbiome. In particular, the present disclosure relates to botanical compositions containing various botanical extracts for modulating the microbiome of the feline gut.

### BACKGROUND

Trillions of microorganisms constitute the gut microbiome in animals, and their presence throughout the gastrointestinal tract play a key role in gut health and immune health. Microorganisms in the gut can include a number of different types of microbes, including bacteria, yeast, fungi, and archaea. When the gut microbiome is out of balance, such as when the relative abundance of one or more benign or beneficial microbes decrease and/or the relative abundance of one or more pathogenic or other detrimental microbes increase past a certain threshold, the imbalances in these microbial populations within the community can result in various adverse health conditions and symptoms for the host animal. Imbalances in microbial populations within the community composition further can lead to imbalances in microbial metabolites, such as volatile fatty acid compounds, that are produced within the gastrointestinal tract by the microbial populations and that further can be indicated as biomarkers of various adverse health conditions.

The feline gut microbiome composition has a broad impact on the gut health, immune function, and mental health of feline animals, as well as impacts on overall lifestyle. Intestinal distress due to alterations in the feline gut microbiome can lead to several undesirable digestive symptoms including vomiting, constipation, and acute or chronic diarrhea. Such symptoms can be indicated in several adverse health conditions associated with alterations in the gut microbiome, including obesity, diarrhea, diabetes, constipation, inflammatory bowel disease, abdominal gas, loss of appetite, poor weight gain, bacterial infection, microbial infection, lethargy, physical and emotional discomfort, and the like. These conditions and symptoms can impact the physical and mental health, and overall wellbeing of feline animals.

Traditional treatments, including the use of antimicrobials, can lead to resistance when such agents are used over long periods of time. Further, existing treatments that include the use of probiotic formulations can be inadequate due to limits on viability of the probiotic organisms during the processing and storage of such products, as well as limits due to degradation of the products throughout the intestinal tract by the time they reach the intended site within the gut. Thus, alternative interventions are needed to address the various health conditions associated with alterations in the gut microbiome.

### SUMMARY

The present disclosure provides a botanical composition including at least one botanical extract. The botanical extract can be present in the botanical composition in an amount effective to produce a beneficial effect on a gut microbiome of an animal. The botanical extract can include one or more botanical extracts including an extract of dandelion leaf, dandelion leaf, ground dandelion leaf, dandelion root, Japanese ginseng, asafoetida, privit, common fennel, Indian mulberry, Rangoon creeper, long pepper, Indian lotus, cayenne/chili pepper, velvet foot winter mushroom, sugar apple, lion's mane mushroom, coriander, saw palmetto, Spanish dagger, mound lily yucca, carob, field lupine, goat's head, sweet prickly pear, partridge berry, bugleweed, Stelmatrocryption khasianum, Amaranthus spinosus, and elderberry, or any combinations thereof.

In an aspect, the botanical extract includes one or more of an extract of sugar apple, velvet foot winter mushroom, or dandelion leaf.

In an aspect, a plurality of botanical extracts are present in the botanical composition, where each botanical extract is present in an amount effective to produce a beneficial effect on the gut microbiome.

In an aspect, the beneficial effect includes one or more of an increase in a relative abundance of one or more beneficial bacterial strains; a decrease in the relative abundance of one or more pathogenic bacterial strains; an increase the Shannon Diversity index; or an increase in volatile fatty acid concentration produced by the gut microbiome.

In an aspect, the increase in the relative abundance of the one or more beneficial bacterial strains includes an increase in a relative abundance of one or more species of Bifidobacterium, Ruminococcaceae, Bacteroidaceae, Faecalibacterium Roseburia, or Prevotella.

In an aspect, the increase in the relative abundance of the one or more beneficial bacterial strains includes an increase in the relative abundance of the one or more beneficial bacterial strains in an animal fed a diet containing the botanical composition as compared to an animal fed a diet lacking the botanical composition.

In an aspect, the increase in the relative abundance of the one or more beneficial bacterial strains includes an increase in the relative abundance of the one or more beneficial bacterial strains in an animal fed a diet containing the botanical composition compared to a relative abundance of the beneficial strains in an animal experiencing one or more adverse health conditions.

In an aspect, the decrease in the one or more pathogenic bacterial strains includes a decrease in the relative abundance of one or more of Burkholderia cepacia, Campylobacter helveticus, Campylobacter jejuni, Clostridium perfringens, Collinsella aerofaciens, Desulfovibrio vulgaris, Enterococcus faecalis, Escherichia coli, Salmonella enterica subsp. enterica serovar Typhimurium, or Streptococcus canis.

In an aspect, the decrease in the relative abundance of the one or more pathogenic bacterial strains includes a decrease in the relative abundance of the one or more pathogenic bacterial strains in an animal fed a diet containing the botanical composition relative to a relative abundance of the pathogenic strains that would cause disease.

In an aspect, the increase in the relative abundance of one or more beneficial bacterial strains includes an increase in a Bifidobacterium population in an animal fed a diet containing the botanical composition as compared to an animal fed a diet lacking the botanical composition.

In an aspect, the increase in the relative abundance of one or more beneficial bacterial strains includes an increase in a Ruminococcaceae population in an animal fed a diet containing the botanical composition as compared to an animal fed a diet lacking the botanical composition.

In an aspect, the increase in the relative abundance of one or more beneficial bacterial strains includes an increase in a Bacteroidaceae population in an animal fed a diet containing the botanical composition as compared to an animal fed a diet lacking the botanical composition.

In an aspect, the increase in the relative abundance of one or more beneficial bacterial strains includes an increase in a Faecalibacterium population in an animal fed a diet containing the botanical composition as compared to an animal fed a diet lacking the botanical composition.

In an aspect, the increase in the volatile fatty acid concentration produced by the gut microbiome includes an increase in the total volatile fatty acid concentration produced by the gut microbiome in an animal fed a diet containing the botanical composition as compared to an animal fed a diet lacking the botanical composition.

In an aspect, the increase in the volatile fatty acids produced by the gut microbiome includes an increase in one or more of acetate, propionate, butyrate, and valerate, or derivatives thereof.

In an aspect, the increase in volatile fatty acid production by the gut microbiome includes an increase in the acetate concentration produced by the gut microbiome in an animal fed a diet containing the botanical composition as compared to an animal fed a diet lacking the botanical composition.

In an aspect, the increase in volatile fatty acid production by the gut microbiome includes an increase of the butyrate concentration produced by the gut microbiome in an animal fed a diet containing the botanical composition as compared to an animal fed a diet lacking the botanical composition.

In an aspect, the increase in volatile fatty acid production by the gut microbiome includes an increase in the propionate concentration produced by the gut microbiome in an animal fed a diet containing the botanical composition as compared to an animal fed a diet lacking the botanical composition.

In an aspect, the animal is a feline animal.

In an aspect, the amount of botanical extract effective to produce a beneficial effect on a gut health of an animal is from 0.0001 wt. % to 10 wt. %, or from 0.01 wt. % to 5.0 wt. %, or from 0.1 wt. % to 1.0 wt. % of a diet of an animal.

The present disclosure provides a method for modulating a gut microbiome of an animal, including administering to the animal a botanical composition including a botanical extract in an amount effective to produce a beneficial effect on a gut health of an animal;
where the amount of botanical extract effective to produce a beneficial effect causes one or more of: measuring the beneficial effect by determining one or more of an increase in a relative abundance of one or more beneficial bacterial strains; a decrease in a relative abundance of one or more pathogenic or detrimental bacterial strains; an increase in a Shannon Diversity index; and a change in a concentration of volatile fatty acid produced by the gut microbiome. The method further can include where the botanical composition includes one or more botanical extracts including an extract of dandelion leaf, dandelion leaf, ground dandelion leaf, dandelion root, Japanese ginseng, asafoetida, privit, common fennel, Indian mulberry, Rangoon creeper, long pepper, Indian lotus, cayenne/chili pepper, velvet foot winter mushroom, sugar apple, lion's mane mushroom, coriander, saw palmetto, Spanish dagger, mound lily yucca, carob, field lupine, goat's head, sweet prickly pear, partridge berry, bugleweed, Stelmatrocryption khasianum, Amaranthus spinosus, and elderberry, or combinations thereof.

In an aspect, the increase in the relative abundance of the one or more beneficial bacterial strains includes an increase in a relative abundance of one or more of Bifidobacterium, Ruminococcaceae, Bacteroidaceae, or Faecalibacterium, Lactobacilluseae, Roseburia, or Prevotella.

In an aspect, the animal is a feline animal.

In an aspect, the amount of botanical extract effective to produce a beneficial effect on a gut microbiome of an animal is from 0.0001 wt. % to 10 wt. %, or from 0.01 wt. % to 5.0 wt. %, or from 0.1 wt. % to 1.0 wt. %.

In an aspect, the one or more pathogenic bacterial strains comprise one or more of Burkholderia cepacia, Campylobacter helveticus, Campylobacter jejuni, Clostridium perfringens, Collinsella aerofaciens, Desulfovibrio vulgaris, Enterococcus faecalis, Escherichia coli, Salmonella enterica subsp. enterica serovar Typhimurium, or Streptococcus canis.

In an aspect, measuring the beneficial effect is determined by comparing the effects observed in an animal administered a diet containing the botanical composition as compared to an animal fed a diet lacking the botanical composition.

In an aspect, the increase in the relative abundance of the one or more beneficial bacterial strains includes an increase in a relative abundance of one or more species of Bifidobacterium, Ruminococcaceae, Bacteroidaceae, or Faecalibacterium.

In an aspect, the increase in the relative abundance of the one or more beneficial bacterial strains includes an increase in the relative abundance of the one or more beneficial bacterial strains in an animal fed a diet containing the botanical composition as compared to an animal In an aspect, the increase in the relative abundance of the one or more beneficial bacterial strains includes an increase in the relative abundance of the one or more beneficial bacterial strains in an animal fed a diet containing the botanical composition compared to a relative abundance of the beneficial strains in an animal experiencing one or more adverse health conditions.

In an aspect, the decrease in the one or more pathogenic bacterial strains includes a decrease in the relative abundance of one or more of Burkholderia cepacia, Campylobacter helveticus, Campylobacter jejuni, Clostridium perfringens, Collinsella aerofaciens, Desulfovibrio vulgaris, Enterococcus faecalis, Escherichia coli, Salmonella enterica subsp. enterica serovar Typhimurium, or Streptococcus canis.

In an aspect, the decrease in the relative abundance of the one or more pathogenic bacterial strains includes a decrease in the relative abundance of the one or more pathogenic bacterial strains in an animal fed a diet containing the botanical composition relative to a relative abundance of the pathogenic strains that would cause disease.

In an aspect, the increase in Bifidobacterium includes an increase in the relative abundance of the Bifidobacterium bacterial population in an animal fed a diet containing the botanical composition as compared to an animal fed a diet lacking the botanical composition.

In an aspect, the increase in Ruminococcaceae includes an increase in the relative abundance of the Ruminococcaceae bacterial population in an animal fed a diet containing the botanical composition as compared to an animal fed a diet lacking the botanical composition.

In an aspect, the increase in Bacteroidaceae includes an increase in the Bacteroidaceae bacterial population in an animal fed a diet containing the botanical composition as compared to an animal fed a diet lacking the botanical composition.

In an aspect, the increase in Faecalibacterium includes an increase in the relative abundance of the Faecalibacterium bacterial population in an animal fed a diet containing the botanical composition as compared to an animal fed a diet lacking the botanical composition.

In an aspect, the change in the volatile fatty acid production by the gut microbiome includes an increase in a total volatile fatty acid concentration produced by the gut microbiome in an animal fed a diet containing the botanical composition as compared to an animal fed a diet lacking the botanical composition.

In an aspect, the change in the concentration of volatile fatty acids by the gut microbiome includes an increase in one or more of acetate, propionate, isobutyrate, butyrate, isovalerate, and valerate, or derivatives thereof.

In an aspect, the change in the concentration of volatile fatty acids is measured in an animal fed a diet containing the botanical composition as compared to an animal fed a diet lacking the botanical composition.

In an aspect, the change in the concentration of volatile fatty acids is measured in an animal in a disease state as compared to a baseline concentration of volatile fatty acids in the animal in a non-diseased state.

In an aspect, the disease state includes diarrhea, obesity, diabetes, inflammatory bowel disease, primary gastrointestinal disease, and severe gastrointestinal disease.

In an aspect, the methods herein further can include reducing one or more symptoms or intestinal distress in the animal, the symptoms including acute diarrhea, chronic diarrhea, constipation, and vomiting.

In an aspect, administering to the animal a botanical composition includes a botanical composition produces at least one health benefit to a feline having one or more health conditions including diarrhea, obesity, diabetes, irritable bowel disease, and primary gastrointestinal disease.

In an aspect, the where at least one health benefit includes cessation of diarrhea, a decrease in obesity, a reduction in diabetic biomarkers, an improvement of irritable bowel disease, or a decrease in incidence of primary gastrointestinal disease.

### BRIEF DESCRIPTION OF THE FIGURES

Not applicable.

### DETAILED DESCRIPTION

Reference will now be made in detail to certain aspects of the disclosed subject matter. While the disclosed subject matter will be described in conjunction with the enumerated claims, it will be understood that the exemplified subject matter is not intended to limit the claims to the disclosed subject matter.

As described above, various adverse health conditions can be correlated with increases or decreases in the relative abundance of various types of microbes within the gut microbiome of an animal. As used herein, the term “microbes” can include, but is not to be limited to, bacteria, yeast, fungi, and archaea. In various aspects, one or more adverse health conditions can be correlated with increases or decreases in the relative abundance of various types of bacteria, yeast, fungi, and archaea, or any combination thereof, within the gut microbiome of an animal. In other aspects, various adverse health conditions can be correlated with increases or decreases in the relative abundance of various types of bacteria within the gut microbiome of an animal. The adverse health conditions can include, but are not to be limited to obesity, diarrhea, diabetes, constipation, inflammatory bowel disease, abdominal gas, loss of appetite, poor weight gain, bacterial infection, lethargy, physical and emotional discomfort, or combinations thereof.

As used herein, the term “relative abundance” refers to the evenness of distribution of an individual species among all species in the total community, or population, of species and can be measured as a function of all microbes or a subset of a give type of microbe.

The relative abundance of an individual species of microbe in a total community of species of microbes can refer to a fraction (i.e., a value between 0 to 1) that the individual species of microbe contributes to the total microbe count in the community. It will be appreciated that the relative abundance of any given microbe can be measured within a total community of species of a single healthy animal, or it further can be measured against a previously measured average relative abundance or a predicted average relative abundance across a population of healthy animals. Similarly, the relative abundance of any given microbe can be measured within a total community of species of a single animal experiencing one or more adverse health conditions, or it further can be measured against a previously measured average relative abundance or a predicted average relative abundance across a population of animals experiencing one or more adverse health conditions.

Imbalances in the relative abundance of the microbial populations that comprise the gut microbiome can include a decrease in the relative abundance of one or more benign or beneficial microbes, and/or an increase in the relative abundance of one or more pathogenic or detrimental microbes. The imbalances in the relative abundance of the microbial populations that comprise the gut microbiome can include a decrease in the relative abundance of one or more benign or beneficial bacterial, yeast, fungal, and archaeal strains, and/or an increase in the relative abundance of one or more pathogenic or detrimental microbes including bacterial, yeast, fungal, and archaeal strains. In various aspects, the imbalances can be measured relative to a predetermined baseline relative abundance for a given microbe. It will be appreciated that a predetermined baseline relative abundance can be an indicator for a healthy community structure in the gut. A healthy community structure in the gut can support digestion and immunity conducive to maintaining a state in an animal free from various adverse health conditions. Imbalances in the microbial populations of the gut microbiome that result in a shift in a relative abundance for given microbe can cause clinical or subclinical symptoms in the animal(s) as associated with one or more adverse health conditions. With a need in the industry to shift from the use of antimicrobials, such as antibiotics, there exists a need to enhance beneficial microbes and reduce pathogens or detrimental microbes by using alternative interventions. In accordance with various aspects herein, botanical compositions and methods for feeding an animal the botanical compositions are included. The botanical compositions and methods herein are configured to modulate various microbial populations in the gut so as to address any imbalances in the gut microbiome and to improve various gut health and immune health measures in the process.

The present disclosure provides botanical compositions that can include at least one botanical extract in an amount effective to produce a beneficial effect on gut health of an animal(s). As used herein, the term “gut health” can refer to the efficient and effective digestion of food by the digestive system (e.g., esophagus, stomach, gall bladder, liver, pancreas, spleen, large intestine (i.e., colon), small intestine, rectum, and anus); the absence of abdominal pain as caused by one or more adverse health conditions; the absence of increased intestinal permeability, mucosal inflammation, excess gas, pH, and a deficiency or excess of volatile fatty acids; or any combinations thereof.

It will be appreciated that gut health and immune health can be interdependent, such that an improvement in gut health can lead to an improvement in immune health, and a deterioration or decrease in gut health can lead to a deterioration or decrease in immune health.

As used herein, the term “immune health” can refer to the standard functioning of the immune system as it is understood, where the immune system includes at least the mucous membranes of the nose, mouth, and throat; the tonsils; the lymph nodes; the thymus; the spleen; the large and small intestines; the bone marrow; the immune cells of the blood, including at least monocytes, lymphocytes, neutrophils, eosinophils, basophils, macrophages, erythrocytes, platelets, stem cells, and the like; and the skin.

As used herein, the term “botanical extracts” refers to extracts isolated from plant sources. Examples of botanical extracts suitable for use in the botanical compositions herein can include, but are not to be limited to, one or more of an extract of dandelion leaf, dandelion leaf, ground dandelion leaf, dandelion root, Japanese ginseng, asafoetida (devil's dung), privit, common fennel, Indian mulberry, Rangoon creeper, long pepper, Indian lotus, cayenne/chili pepper, velvet foot winter mushroom, sugar apple, lion's mane mushroom, coriander, saw palmetto, Spanish dagger, mound lily yucca, carob, field lupine, goat's head, sweet prickly pear, partridge berry, bugleweed, Stelmatrocryption khasianum (Sheng Teng), Amaranthus spinosus (spiny amaranth), and elderberry, or combinations thereof.

The term “volatile fatty acids” as used herein can include, but is not to be limited to, short-chain fatty acids including those having from C2-C6 carbon atoms and that are carboxylic acids produced during the anaerobic digestion process of one or more microorganisms. The short-chain fatty acids can include, but are not to be limited to, acetic acid, propionic acid, butyric acid, isobutyric acid, valeric acid, isovaleric acid, 2-methylbutyric acid, and hexanoic acid. It will be appreciated that the anionic conjugate base form of the short-chain fatty acids recited herein include acetate, propionate, butyrate, isobutyrate, valerate, isovalerate, and 2-methylbutyrate, hexanoate, respectively. The anionic conjugate base form of the short-chain fatty acids herein can be present in a balanced salt form having associated therewith any one of the alkali metals or alkaline earth metals including sodium, potassium, magnesium, and the like. Unless otherwise noted, the volatile fatty acids as referred to herein can include any form, including one or more of the acid form, the anionic form, or in the salt form. It will be appreciated that the form that the volatile fatty acid takes on will be based on the pH of the gut environment where it is produced.

As used herein, a “beneficial effect” can refer to an effect that the botanical compositions herein have on the gut microbiome of an animal that can manifest as one or more improvements in gut health and immune health of the animal, and can include increasing the efficiency and effectiveness of digestion of food; reducing or preventing abdominal pain as caused by one or more adverse health conditions; reducing or preventing an adverse health condition(s); reducing or preventing an increased intestinal permeability or mucosal inflammation; and a balancing of any deficiency in or excess of volatile fatty acids; or any combinations thereof. A beneficial effect can be determined by one or more of an increase in a relative abundance of one or more beneficial microbial strains; a decrease in a relative abundance of one or more pathogenic or detrimental microbial strains; an increase in a relative abundance of one or more beneficial bacterial strains; a decrease in a relative abundance of one or more pathogenic or detrimental bacterial strains; an increase in a relative abundance of one or more beneficial yeast strains; a decrease in a relative abundance of one or more pathogenic or detrimental yeast strains; an increase in a relative abundance of one or more beneficial fungal strains; a decrease in a relative abundance of one or more pathogenic or detrimental fungal strains; an increase in a relative abundance of one or more beneficial archaeal strains; a decrease in a relative abundance of one or more pathogenic or detrimental archaeal strains; an increase in a Shannon Diversity index; and a change in a concentration of volatile fatty acid produced by the gut microbiome, as described elsewhere herein. The beneficial effect can be measured in an animal fed a diet containing the botanical composition as compared to an animal fed a diet lacking the botanical composition. The beneficial effect further can be measured in a healthy animal fed a diet containing the botanical composition as compared to an animal experiencing an adverse health condition and fed a diet lacking the botanical composition. The beneficial effect further can be measured in an animal experiencing an adverse health condition and fed a diet containing the botanical composition as compared to an animal experiencing an adverse health condition and fed a diet lacking the botanical composition. The beneficial effect further can be measured in an animal experiencing an adverse health condition and fed a diet containing the botanical composition as compared to a healthy animal fed a diet lacking the botanical composition.

The present disclosure provides botanical compositions having an amount of botanical extract effective to produce a beneficial effect on the gut health of an animal by modulating the microbial populations of the gut microbiome. The botanical compositions herein can be administered to an animal in an amount effective to produce a beneficial effect that can result in the prevention of, an improvement of, or the reversal of one or more adverse health conditions including obesity, diarrhea, diabetes, constipation, inflammatory bowel disease, abdominal gas, loss of appetite, poor weight gain, bacterial infection, microbial infection, lethargy, physical and emotional discomfort. Modulation of the microbial populations of the gut microbiome can be accomplished through the administration of botanical compositions described herein. The modulation of the microbial populations can result from the inhibition of growth of one or more pathogenic or otherwise detrimental types of microbes. The modulation of the microbial populations further can result from the promotion of growth of one or more beneficial types of microbes. By way of example, the modulation of the microbial populations can result from the inhibition of growth of one or more pathogenic or otherwise detrimental types of bacteria, yeast, fungi, and archaea; or the promotion of growth of one or more beneficial types of bacteria, yeast, fungi, and archaea. In various aspects, the modulation of the microbial populations can result from the inhibition of growth of one or more pathogenic or otherwise detrimental bacterial populations; or the promotion of growth of one or more beneficial bacterial populations.

The present disclosure provides that the botanical compositions herein can include one or more botanical extracts in an amount effective to produce an inhibitory effect on one or more pathogenic or detrimental microbes. The inhibitory effect can include reducing or preventing an infection with the one or more pathogenic or detrimental microbes through growth inhibition. In various aspects, the amount of one or more botanical extracts effective to produce an inhibitory effect on one or more pathogenic or detrimental microbes can produce an inhibitory effect including reducing or preventing an infection with the one or more pathogenic or detrimental microbes through growth inhibition. The effective amount of one or more botanical extracts in the diet can directly or indirectly inhibit the growth of the one or more pathogenic or detrimental microbes and can confer one or more beneficial effects to the animal(s). By way of example, an effective amount of one or more botanical extracts in the diet can directly or indirectly inhibit the growth of the one or more pathogenic or detrimental microbes and can confer one or more beneficial effects to the animal(s) by decreasing the relative abundance of the pathogenic or detrimental microbes present in the gut microbiome. The present disclosure further provides the botanical compositions containing one or more botanical extracts in an amount effective to increase the relative abundance of one or more beneficial microbes through growth promotion. The amount of one or more botanical extracts suitable for use in the botanical compositions herein can be effective to directly or indirectly promote the growth of the one or more beneficial microbes and can confer one or more beneficial effects to the animal(s). In some aspects, the present disclosure provides the botanical compositions containing one or more botanical extracts in an amount effective to both reduce or prevent the growth of pathogenic or detrimental microbes and promote the growth of beneficial microbes. It will be appreciated that the microbes can include any of bacteria, yeast, fungi, or archaea.

Examples of botanical extracts suitable for use in the botanical compositions herein can include, but are not to be limited to, one or more of an extract of dandelion leaf, dandelion leaf, ground dandelion leaf, dandelion root, Japanese ginseng, asafoetida (devil's dung), privit, common fennel, Indian mulberry, Rangoon creeper, long pepper, Indian lotus, cayenne/chili pepper, velvet foot winter mushroom, sugar apple, lion's mane mushroom, coriander, saw palmetto, Spanish dagger, mound lily yucca, carob, field lupine, goat's head, sweet prickly pear, partridge berry, bugleweed, Stelmatrocryption khasianum (Sheng Teng), Amaranthus spinosus (spiny amaranth), and elderberry, or combinations thereof. In some aspects, the suitable botanicals can include one or more of an extract of sugar apple, velvet foot winter mushroom, and dandelion leaf, or any combination thereof. It will be appreciated that in some aspects, the botanical composition only contains one or more botanical extract.

### Botanical Compositions

The botanical compositions herein can be included in the diet of an animal as a supplement to any complete and balanced food products or can be provided as an ingredient of a food product. The botanical compositions herein can be administered directly to any suitable animal species or can be administered to the animal as a component of a food product or supplement product suitable for a variety of species. In some aspects, the animal is a feline animal. In various aspects, the food products referred to herein can include a complete and balanced food product suitable for a variety of species, including felines. In some aspects, the complete food product is a feline food product. A complete feline food product can include a nutritionally complete and balanced daily dietary composition that is fed as the sole ration and can maintain life, promote growth, and sustain reproduction without any additional substances being consumed except water. Complete feline food products can include mixtures containing appropriate levels of the nutrients required to sustain the life of the felines, provided among them proteins, fats, carbohydrates, and the like.

The botanical compositions herein can include one or more botanical extracts. The botanical extract can be present in the botanical compositions in an amount effective to produce one or more beneficial effects on the gut microbiome of an animal. In various aspects, the botanical extracts can include one or more of an extract of dandelion leaf, dandelion leaf, ground dandelion leaf, dandelion root, Japanese ginseng, asafoetida (devil's dung), privit, common fennel, Indian mulberry, Rangoon creeper, long pepper, Indian lotus, cayenne/chili pepper, velvet foot winter mushroom, sugar apple, lion's mane mushroom, coriander, saw palmetto, Spanish dagger, mound lily yucca, carob, field lupine, goat's head, sweet prickly pear, partridge berry, bugleweed, Stelmatrocryption khasianum (Sheng Teng), Amaranthus spinosus (spiny amaranth), and elderberry, or combinations thereof. In some aspects, the suitable botanicals can include one or more of an extract of sugar apple, and velvet foot winter mushroom, or any combination thereof. In various aspects, the botanical compositions can include a plurality of botanical extracts, where each botanical extract is independently or collectively present in an amount effective to produce a beneficial effect on the gut microbiome.

The amount of botanical extract effective to produce a beneficial effect on the gut health of an animal can include from 0.0001 wt. % to 10 wt. %, or from 0.01 wt. % to 5.0 wt. %, or from 0.1 wt. % to 1.0 wt. % of a diet of an animal. The amount of one or more botanical extracts effective to produce a beneficial effect on the gut health of an animal can be from 0.0001 wt. %, 0.001 wt. %, 0.01 wt. %, 0.1 wt. %, 0.2 wt. %, 0.3 wt. %, 0.4 wt. %, 0.5 wt. %, 0.6 wt. %, 0.7 wt. %, 0.8 wt. %, 0.9 wt. %, 1.0 wt. %, 1.25 wt. %, 1.5 wt. %, 1.75 wt. %, 2.0 wt. %, 2.25 wt. %, 2.5 wt. %, 2.75 wt. %, 3.0 wt. %, 3.25 wt. %, 3.5 wt. %, 3.75 wt. %, 4.0 wt. %, 4.25 wt. %, 4.5 wt. %, 4.75 wt. %, 5.0 wt. %, 6.0 wt. %, 7.0 wt. %, 8.0 wt. %, 9.0 wt. %, or 10 wt. % of the diet, or it can be any amount falling within a range of any of the forgoing.

In addition to the botanical extract, the botanical compositions herein further can include various ingredients to suitable for a feline diet, including, but not to be limited to, meats including poultry such as chicken, turkey, geese, duck, ostrich, quail, and pheasant; beef; buffalo; pork; lamb; venison; fish, including but not limited to whitefish, cod, pollock, salmon, and tuna; crustaceans; liver; or animal byproducts. The botanical compositions herein further can include grains such as wheat, rice, oats, soy, other cereals, and flours thereof. The botanical compositions can also include various fruits and vegetables, including but not to be limited to, sweet potato, carrot, spinach, potato, pea, pumpkin, beets, squash, green leafy vegetables, apples, berries, and corn. The botanical compositions herein further can include seeds or legumes, including, but not to be limited to chickpeas and chickpea flour, black beans, white beans, kidney beans, sunflower seeds, chia seeds and meals, flax seeds and meals, pea and pea flour, soybeans, grain derived glutens sourced from oats, barley, rice, corn, wheat, and the like. The botanical compositions further can include various oils, including but not to be limited to, vegetable oils, sunflower oil, safflower oil, canola oil, soybean oil, olive oil, and coconut oil, and the like. The botanical compositions herein further can include herbs, natural and artificial flavors, spices, extracts, glycerin, tapioca starch, soy lecithin, sunflower lecithin, and natural preservatives. In various aspects, the botanical compositions herein do not include inulin.

The total protein in the botanical compositions can be from about 5 wt. % to about 75 wt. % on a dry matter basis (DM basis), from about 10 wt. % to about 50 wt. % on a DM basis, from about 20 wt. % to about 45 wt. % on a DM basis, or from about 16 wt. % to about 26 wt. % on a DM basis. Total protein in the botanical compositions can be from 5 wt. %, 10 wt. %, 15 wt. %, 20 wt. %, 25 wt. %, 30 wt. %, 35 wt. %, 40 wt. %, 45 wt. %, 50 wt. %, 55 wt. %, 60 wt. %, 65 wt. %, 70 wt. %, or 75 wt. % of the botanical composition on a DM basis or can be any amount falling within a range of any of the forgoing. The total protein in the botanical compositions can be variable depending on the formulation and intended use of the botanical composition. For example, a botanical composition formulated for a kitten can include from about 30 wt. % to about 50.0% protein on a DM basis. A botanical composition formulated for an adult cat can include from about 26 wt. % to about 75.0 wt. % protein on a DM basis.

Total fat (e.g., oil, fat, and/or lipids) in the botanical compositions can be from about 2 wt. % to about 50 wt. % on a DM basis, from about 10 wt. % to about 30 wt. % on a DM basis, or from about 15 wt. % to about 25 wt. % on a DM basis. Total fat in the botanical compositions can be from 2.0 wt. %, 2.5 wt. %, 5.0 wt. %, 5.25 wt. %, 5.5%, 5.75 wt. %, 6.0 wt. %, 6.5 wt. %, 7.0 wt. %, 7.5 wt. %, 8.0 wt. %, 8.5 wt. %, 9.0 wt. %, 9.5 wt. %, 10.0 wt. %, 12.0 wt. %, 13.0 wt. %, 14.0 wt. %, 15.0 wt. %, 16.0 wt. %, 17.0 wt. %, 18.0 wt. %, 19.0 wt. %, 20.0 wt. %, 30.0 wt. %, 40.0 wt. %, or 50.0 wt. % of the botanical composition on a DM basis, or can be any amount falling within a range of any of the forgoing. The total fat in the botanical composition can be variable depending on the formulation and intended use of the botanical composition. For example, a botanical composition formulated for a kitten can include from about 9% to about 30.0% fat on a DM basis. A botanical composition formulated for an adult cat can include from about 9% to about 50.0% fat on a DM basis. It will be appreciated that fats suitable for feline consumption can include linoleic acid, alpha-linoleic acid, arachidonic acid, eicosapentaenoic acid, and docosahexaenoic acid.

The total carbohydrate in the botanical composition can be from about 0 wt. % to 75 wt. % on a DM basis, from about 10 wt. % to about 50 wt. % on a DM basis, from about 20 wt. % to about 45 wt. % on a DM basis. Total carbohydrate in the botanical composition can be from 0 wt. %, 2.5 wt. %, 5 wt. %, 10 wt. %, 15 wt. %, 20 wt. %, 25 wt. %, 30 wt. %, 35 wt. %, 40 wt. %, 45 wt. %, 50 wt. %, 55 wt. %, 60 wt. %, 65 wt. %, 70 wt. %, or 75 wt. % of the botanical composition on a DM basis or can be any amount falling within a range of any of the forgoing. The total carbohydrate in the botanical composition can be variable depending on the formulation and intended use of the botanical composition.

The botanical compositions herein can be configured such that water can be added to the botanical composition prior to administering it to the animals. The botanical composition herein can be prepared in a wet formulation (about 35 wt. % to 80 wt. % water content), a semi-moist formulation (about 15 wt. % to 30 wt. % water content), or a dry formulation (about 6 wt. % to 10 wt. % water content).

In addition, vitamins and minerals can be added to the botanical composition herein. Additional ingredients can include, but are not to be limited to vitamin A (retinyl-acetate), vitamin D3 (cholecalciferol), vitamin E (DL-α-tocopherol), vitamin K3 (menadione), vitamin B1 (thiamine), vitamin B2 (riboflavin), vitamin B3 (niacin), vitamin B5 (pantothenic acid), vitamin B6 (pyridoxine-HCL), vitamin B7 (biotin), vitamin B9 (folic acid), vitamin B12 (cyanocobalamin)), minerals (e.g., calcium, phosphorus, potassium, sodium, chloride, magnesium, iron, copper, manganese, zinc, iodine, selenium, monocalcium phosphate (CaH₄P₂O₈), sodium bicarbonate (NaHCO₃), sodium chloride (NaCl), potassium chloride (KCl), potassium iodide (KI)) amino acids (e.g., arginine, histidine, isoleucine, leucine, lysine, methionine, methionine-cystine, phenylalanine, phenylalanine-tyrosine, threonine, tryptophan, valine, taurine), ferrous (II) sulfate monohydrate (FeSO₄·H₂O); cupric sulfate (CuSO₄·5H₂O); manganese (II) oxide (MnO); MnSO₄·H₂O; zinc sulfate (ZnSO₄·H₂O) sodium selenite (Na₂SeO₃), choline chloride (C₅H₁₄ClNO), enzymes, and various combinations thereof. In various aspects, ferrous (II) sulfate monohydrate (FeSO₄·H₂O); cupric sulfate (CuSO₄·5H₂O); manganese (II) oxide (MnO); MnSO₄·H₂O; zinc sulfate (ZnSO₄·H₂O; Na₂SeO₃), choline chloride (C₅H₁₄ClNO), enzymes, can be included in chelated mineral forms. By way of example, chelated minerals can include those that are chelated to one or more type of amino acid to facilitate absorption and uptake by the animal.

The botanical compositions herein can be in any suitable form, including a dry solid, a semi-solid form, or a liquid. For example, the botanical composition can be a solid composition in the form of granules, flakes, pellets, powders, tablets, pills, capsules, cubes, chews, crumbles, pastes, gels, and the like. The botanical compositions suitable for use herein can be designed to be mixed with a feline food product or can be fed directly to feline animals. In various aspects, the botanical composition can be a dry solid. In some aspects, the botanical composition can be administered to a feline animal as a top dress applied to a daily food ration or meal. In other aspects, the botanical compositions can be a wet solid. In some aspects, the botanical composition can be a liquid, such as a liquid spray, a liquid water additive, a liquid drench, or a liquid water dip bath for application to a food product. It will be appreciated that the liquid drench as described herein can include one or more of a suspension, a solution, or an emulsion. In various aspects the botanical compositions herein can be in a suspension with one or more oils, including but not to be limited to, vegetable oils, sunflower oil, safflower oil, canola oil, soybean oil, olive oil, and coconut oil, and the like. In some aspects, the botanical compositions herein can include a mixture of a solid and liquid component. The botanical composition can be suitable for administration in the diet of various species of felines as described in more detail below.

In various aspects, the botanical compositions herein can be formed into many shapes and sizes. In various aspects, the botanical compositions herein can be in the shape of a triangle, a square, a rectangle, a sphere, a diamond, a heart, a pellet, a clover, a flower, a fish, a vegetable, a star, an amorphous shape, and the like. The botanical compositions can be formed by a process including one or more of extrusion, retort, cold-pressing, high-pressure processing, and the like.

### Methods for Producing a Beneficial Effect

The present disclosure provides methods for feeding an animal the botanical compositions described herein to produce a beneficial effect on the gut microbiome of an animal. The methods herein can include administering the botanical compositions to an animal having an adverse health condition. The methods further can include administering the botanical compositions to an animal to prevent an adverse health condition. The methods for feeding an animal can include administering to an animal or group of animals the botanical compositions including at least one botanical extract in an amount effective to produce a beneficial effect on a gut health. In various aspects, the methods herein can be used to feed and administer the botanical compositions to feline animals.

The beneficial effects described herein can be determined as an effect exerted by the botanical compositions on the gut health of an animal that can be manifest as one or more improvements related to increasing the efficiency and effectiveness of digesting of food; reducing or preventing abdominal pain as caused by one or more adverse health conditions; preventing one or more adverse health conditions; reducing or preventing an increase intestinal permeability, mucosal inflammation, and a deficiency or excess volatile fatty acids; or any combinations thereof. The beneficial effect further can be determined as enhanced gut health or immune health as a direct result or indirect result, or both, of the effect that the botanical compositions have on the gut health of an animal. It will be appreciated that the beneficial effects described herein can be observed in animals having clinical or subclinical symptoms of an adverse health condition. It will further be appreciated that in various aspects, animals that are not experiencing one or more adverse health conditions can also exhibit a beneficial effect when fed the botanical compositions.

The beneficial effects described herein can be determined several ways using the methods described herein. The beneficial effects described herein can be determined by comparing the effects observed in an animal administered a diet containing a botanical composition as compared to an animal fed a diet lacking the botanical composition. In some aspects, the beneficial effect is determined by comparing the effects observed in an animal administered a diet containing the botanical composition as compared to a different period of time when the animal was administered a diet lacking the botanical composition. In various aspects, the beneficial effect is determined by comparing the effects observed in an animal administered a diet containing the botanical composition as compared to a representative population of healthy animals not afflicted with any adverse health conditions. The methods herein further can include producing a beneficial effect in an animal that can be a result of the direct or indirect effects of the botanical compositions on the gut health of an animal.

The methods herein provide an amount of botanical effective to produce a beneficial effect that causes of one or more of an increase in a relative abundance of one or more beneficial microbes; a decrease in the relative abundance of one or more of pathogenic or detrimental bacterial strains; an increase in the Shannon Diversity index; or an increase in volatile fatty acid concentration as produced by the gut microbiome. The one or more beneficial microbes can include, but is not to be limited to, one or more beneficial strains belonging to the families or genera including Bifidobacterium, Ruminococcaceae, Bacteroidaceae, Faecalibacterium, Lactobacilluseae, Roseburia, or Prevotella. The one or more pathogenic or detrimental microbes can include one or more pathogenic or detrimental microbes, including, but not to be limited to, one or more of Burkholderia cepacia, Campylobacter helveticus, Campylobacter jejuni, Clostridium perfringens, Collinsella aerofaciens, Desulfovibrio vulgaris, Enterococcus faecalis, Escherichia coli, Salmonella enterica subsp. enterica serovar Typhimurium, Streptococcus canis, one or more strains of Enterobacteriaceae, one or more strains of Peptostreptococcaceae, one or more strains of Brucella, or one or more strains of Blautia. It will be appreciated that the microbes can include any of bacteria, yeast, fungi, or archaea.

### Increasing Beneficial Microbes

The methods herein can include determining a beneficial effect, where the beneficial effect is a result of an increase in the relative abundance of the one or more beneficial microbes. In various aspects, the one or more beneficial microbes include one or more beneficial bacterial strains. It will be appreciated that the beneficial microbes can include any of bacteria, yeast, fungi, or archaea. In various aspects, the botanical compositions herein can modulate the beneficial bacterial strains within the gut microbiome of an animal. The botanical compositions herein can lead to a beneficial effect resulting in an increase in a relative abundance of one or more beneficial bacterial strains, such as one or more strains including Bifidobacterium, Ruminococcaceae, Bacteroidaceae, Faecalibacterium, Lactobacilluseae, Roseburia, or Prevotella. In an aspect, the beneficial effect can be determined by an increase in the relative abundance of beneficial bacteria in the gut of an animal. The increase in the relative abundance of the beneficial bacterial strain can modulate the production of volatile fatty acids by the gut microbiome. It will be appreciated that the beneficial effect can be measured as an increase in beneficial bacterial strains with a concomitant decrease in pathogenic or detrimental bacteria.

The increase in the relative abundance of the beneficial bacteria in the gut of an animal can be measured as an increase in the relative abundance of the one or more beneficial bacterial strains in an animal fed a diet containing the botanical composition as compared to a relative abundance of the beneficial bacteria in the animal prior to administration of the botanical compositions. In various aspects, the comparison is made between healthy animals fed a diet containing the botanical composition to healthy animals fed a diet lacking the botanical composition. In some aspects, the comparison is made between animals experiencing an adverse health condition and fed a diet containing the botanical composition to animals experiencing an adverse health condition and fed a diet lacking the botanical composition. In yet other aspects, the comparison is made between animals experiencing an adverse health condition and fed a diet containing the botanical composition to healthy animals fed a diet lacking the botanical composition. In even further aspects, the comparison is made between healthy animals fed a diet containing the botanical composition to animals experiencing an adverse health condition and fed a diet lacking the botanical composition.

In various aspects, the increase in the relative abundance of one or more beneficial bacterial strains can be measured as an increase in the relative abundance of one or more beneficial bacterial strains in an animal fed a diet containing the botanical composition as compared to the same animal fed a diet lacking the botanical composition. In various aspects, the increase in the relative abundance of one or more beneficial bacterial strains can be measured as an increase in the relative abundance of one or more beneficial bacterial strains in an animal fed a diet containing the botanical composition as compared to a different animal fed a diet lacking the botanical composition. In various aspects, the increase in the relative abundance of one or more beneficial bacterial strains can include an increase in the relative abundance of one or more beneficial bacterial strains in an animal fed a diet containing the botanical composition as compared to an average value for the relative abundance of one or more beneficial bacterial strains in a representative population of animals afflicted with one or more adverse health conditions. In various aspects, the increase in the relative abundance of one or more beneficial bacterial strains can be measured as an increase in the relative abundance of one or more beneficial bacterial strains in an animal fed a diet containing the botanical composition as compared to a different point in time when that same animal is fed a diet lacking the botanical composition. In some aspects, the different point in time can include a time when the animal is experiencing an adverse health condition and not consuming the botanical composition.

The increase in the relative abundance of one or more beneficial bacterial strains can include an increase in the relative abundance of one or more strains belonging to the families or genera of Bifidobacterium, Ruminococcaceae, Bacteroidaceae, Faecalibacterium, Lactobacillusceae, Roseburia, or Prevotella, or any combination thereof. In various aspects, the increase in the relative abundance of one or more beneficial bacterial strains can include an increase in a Bifidobacterium relative abundance in an animal fed a diet containing the botanical composition as compared to an animal fed a diet lacking the botanical composition. In various aspects, the increase in the relative abundance of one or more beneficial bacterial strains can include an increase in a Ruminococcaceae relative abundance in an animal fed a diet containing the botanical composition as compared to an animal fed a diet lacking the botanical composition. In various aspects, the increase in the relative abundance of one or more beneficial bacterial strains can include an increase in a Bacteroidaceae relative abundance in an animal fed a diet containing the botanical composition as compared to an animal fed a diet lacking the botanical composition. In various aspects, the increase in the relative abundance of one or more beneficial bacterial strains can include an increase in a Faecalibacterium relative abundance in an animal fed a diet containing the botanical composition as compared to an animal fed a diet lacking the botanical composition. In various aspects, the increase in the relative abundance of one or more beneficial bacterial strains can include an increase in a Lactobacilluseae relative abundance in an animal fed a diet containing the botanical composition as compared to an animal fed a diet lacking the botanical composition. In various aspects, the increase in the relative abundance of one or more beneficial bacterial strains can include an increase in a Roseburia relative abundance in an animal fed a diet containing the botanical composition as compared to an animal fed a diet lacking the botanical composition. In various aspects, the increase in the relative abundance of one or more beneficial bacterial strains can include an increase in a Prevotella relative abundance in an animal fed a diet containing the botanical composition as compared to an animal fed a diet lacking the botanical composition.

### Decreasing Pathogenic or Detrimental Microbes

The methods herein can include determining a beneficial effect, where the beneficial effect is a result of a modulation of the relative abundance of the one or more pathogenic or detrimental microbes. It will be appreciated that the pathogenic or detrimental microbes can include any of bacteria, yeast, fungi, or archaea. In various aspects, the one or more pathogenic or detrimental microbes include one or more pathogenic or detrimental bacterial strains. In various aspects, the botanical compositions herein can lead to a beneficial effect by resulting in a decrease in the relative abundance of pathogenic or detrimental bacterial strains within the gut microbiome of an animal. The botanical compositions herein can lead to a beneficial effect as a result of a decrease in the relative abundance of one or more pathogenic or detrimental bacterial strains, including a decrease in one or more strains of Burkholderia cepacia, Campylobacter helveticus, Campylobacter jejuni, Clostridium perfringens, Collinsella aerofaciens, Desulfovibrio vulgaris, Enterococcus faecalis, Escherichia coli, Salmonella enterica subsp. enterica serovar Typhimurium, or Streptococcus canis. In an aspect, the beneficial effect can be measured as a decrease in pathogenic or detrimental bacterial strains in the gut of an animal. The decrease in the relative abundance of the pathogenic or detrimental bacterial strains can modulate the production of volatile fatty acids by the gut microbiome. It will be appreciated that the beneficial effect can be measured as a decrease in pathogenic or detrimental bacterial strains with a concomitant increase in beneficial bacteria.

The decrease in the pathogenic or detrimental bacteria in the gut of an animal can be measured as a decrease in the relative abundance of the one or more pathogenic or detrimental strains in an animal fed a diet containing the botanical composition as compared to a relative abundance in the animal prior to administration of the botanical compositions. In various aspects, the comparison is made between healthy animals fed a diet containing the botanical composition to healthy animals fed a diet lacking the botanical composition. In some aspects, the comparison is made between animals experiencing an adverse health condition and fed a diet containing the botanical composition to animals experiencing an adverse health condition and fed a diet lacking the botanical composition. In yet other aspects, the comparison is made between animals experiencing an adverse health condition and fed a diet containing the botanical composition to healthy animals fed a diet lacking the botanical composition. In even further aspects, the comparison is made between healthy animals fed a diet containing the botanical composition to animals experiencing an adverse health condition and fed a diet lacking the botanical composition.

In various aspects, the decrease in the relative abundance of one or more pathogenic or detrimental bacteria can be measured as a decrease in the relative abundance of one or more pathogenic or detrimental bacterial strains in an animal fed a diet containing the botanical composition as compared to the same animal fed a diet lacking the botanical composition. In various aspects, the decrease in the relative abundance of one or more pathogenic or detrimental bacterial strains can be measured as a decrease in the relative abundance of one or more pathogenic or detrimental bacterial strains in an animal fed a diet containing the botanical composition as compared to a different animal fed a diet lacking the botanical composition. In various aspects, the decrease in the relative abundance of one or more pathogenic or detrimental bacterial strains can include a decrease in the relative abundance of one or more pathogenic or detrimental bacterial strains in an animal fed a diet containing the botanical composition as compared to an average value for the relative abundance of one or more pathogenic or detrimental bacterial strains in a representative population of animals afflicted with one or more adverse health conditions. In various aspects, the decrease in the relative abundance of one or more pathogenic or detrimental bacterial strains can be measured as a decrease in the relative abundance of one or more pathogenic or detrimental bacterial strains in an animal fed a diet containing the botanical composition as compared to a different point in time when that same animal is fed a diet lacking the botanical composition. In some aspects, the different point in time can include a time when the animal is experiencing an adverse health condition and not consuming the botanical composition.

### Shannon Diversity Index

The methods herein can include determining a beneficial effect where the beneficial effect is a result of an increase in the Shannon Diversity Index. In various aspects, administration of the botanical compositions herein can affect the value for the Shannon Diversity Index. Without wishing to be bound by any particular theory, it is believed that the Shannon Diversity Index is a measure of species diversity within a population. The Shannon Diversity Index, H, can be calculated as:

H=−Σpᵢ×ln(pᵢ)
where Σ indicates summation, pᵢ indicates the proportion of a population made up of species i, and ln (pᵢ) indicates the natural log of the proportion of the population made up of species i. In various aspects, the higher the value for H, the higher the diversity of species in a population, such as in a bacterial population. Conversely, the lower the value for H, the lower the diversity of species in a population. In various aspects herein, an increase in the Shannon Diversity Index can be measured as an increase in the Shannon Diversity Index in an animal fed a diet containing the botanical composition as compared to an animal fed a diet lacking the botanical composition. In various aspects, the increase in the Shannon Diversity Index can be measured as an increase in the Shannon Diversity Index in an animal fed a diet containing the botanical composition as compared to an average value for the Shannon Diversity Index in a representative population of animals afflicted with one or more adverse health conditions. In various aspects, an increase in the Shannon Diversity Index can be measured as an increase in the Shannon Diversity Index in an animal fed a diet containing the botanical composition as compared to a different point in time when that same animal is fed a diet lacking the botanical. In some aspects, the different point in time can include a time when the animal is experiencing an adverse health condition and not consuming the botanical composition.

### Modulation of Volatile Fatty Acids

The methods herein can include determining a beneficial effect where the beneficial effect is a result of modulation in the volatile fatty acid production by the gut microbiome. Modulation of the microbial populations of the gut microbiome further can result in increases or decreases in various microbial metabolites within the gut, including but not to be limited to, volatile fatty acids. Modulation of various volatile fatty acids can include an increase or a decrease in one or more of acetic acid, propionic acid, butyric acid, isobutyric acid, valeric acid, isovaleric acid, 2-methylbutyric acid, and hexanoic acid; or the anionic forms, salt forms, or derivatives thereof. In various aspects, an increase in the volatile fatty acid concentration produced by the gut microbiome can be measured as an increase in the volatile fatty acid concentration produced by the gut microbiome in an animal fed a diet containing the botanical composition as compared to an animal fed a diet lacking the botanical composition. In various aspects, the increase in the volatile fatty acid concentration produced by the gut microbiome can be measured as an increase in the volatile fatty acid concentration produced by the gut microbiome in an animal fed a diet containing the botanical composition as compared to an average value for the volatile fatty acid concentration produced by the gut microbiome in a representative population of animals afflicted with one or more adverse health conditions. In various aspects, the increase in the volatile fatty acid concentration produced by the gut microbiome can be measured as an increase in the volatile fatty acid concentration produced by the gut microbiome in an animal fed a diet containing the botanical composition as compared to a different point in time when that same animal is fed a diet lacking the botanical composition. In some aspects, the different point in time can include a time when the animal is experiencing an adverse health condition and not consuming the botanical composition.

It will further be appreciated that a decrease in the volatile fatty acid concentration produced by the gut microbiome can be measured as a decrease in the volatile fatty acid concentration produced by the gut microbiome in an animal fed a diet containing the botanical composition as compared to an animal fed a diet lacking the botanical composition. In various aspects, a decrease in the volatile fatty acid concentration produced by the gut microbiome can be measured as a decrease in the volatile fatty acid concentration produced by the gut microbiome in an animal fed a diet containing the botanical composition as compared to an average value for the volatile fatty acid concentration produced by the gut microbiome in a representative population of animals afflicted with one or more adverse health conditions. In various aspects, a decrease in the volatile fatty acid concentration produced by the gut microbiome can be measured as a decrease in the volatile fatty acid concentration produced by the gut microbiome in an animal fed a diet containing the botanical composition as compared to a different point in time when that same animal is fed a diet lacking the botanical composition. In some aspects, the different point in time can include a time when the animal is experiencing an adverse health condition and not consuming the botanical composition.

In various aspects, an increase in the volatile fatty acids produced by the gut microbiome can include an increase in one or more of acetate, propionate, butyrate, and valerate, or derivatives thereof. In some aspects, the increase in the volatile fatty acid concentration produced by the gut microbiome includes an increase in the total volatile fatty acid concentration produced by the gut microbiome in an animal fed a diet containing the botanical composition as compared to an animal fed a diet lacking the botanical composition. In some aspects, the increase in the volatile fatty acid concentration produced by the gut microbiome includes an increase in the acetate concentration produced by the gut microbiome in an animal fed a diet containing the botanical composition as compared to an animal fed a diet lacking the botanical composition. In some aspects, the increase in the volatile fatty acid concentration produced by the gut microbiome includes an increase in the butyrate concentration produced by the gut microbiome in an animal fed a diet containing the botanical composition as compared to an animal fed a diet lacking the botanical composition. In some aspects, the increase in the volatile fatty acid concentration produced by the gut microbiome includes an increase in the propionate concentration produced by the gut microbiome in an animal fed a diet containing the botanical composition as compared to an animal fed a diet lacking the botanical composition.

The methods herein can include modulating a gut microbiome in an animal with an adverse health condition, including obesity, diarrhea, diabetes, constipation, inflammatory bowel disease, abdominal gas, loss of appetite, poor weight gain, bacterial infection, lethargy, physical and emotional discomfort, and the like. The methods can include administering to the animal a botanical composition including a botanical extract to an animal having an adverse health condition in an amount effective to produce a beneficial effect on a gut health of an animal. The methods can include measuring one or more beneficial effects in an animal. The one or more measured beneficial effects can be indicative of the improvement or cessation of one or more adverse health conditions. In various aspects, the animals fed and administered the botanical compositions herein do not have an adverse health condition.

Feeding an animal with the botanical compositions herein can begin upon birth or upon weaning and can extend throughout an animal's lifecycle. Feeding animals the botanical compositions described herein can be performed over a discrete time span or life stage and can be performed at multiple discrete time spans or life stages during an animal's life cycle. It will be appreciated that the botanical compositions herein can be administered as a daily botanical composition that is fed to the animals on most days or on all days. In various aspects, the botanical composition can be administered to animals on one, two, three, four, five, six, or seven days of the week for a predetermined time span or for the life of the animal. In some aspects, the botanical composition can be adminstered to animals once a day or twice a day.

The botanical compositions herein can be fed to the animal(s) during any of its discrete life stages, throughout the lifespan of the animal, or only when the animal has an active imbalance in the gut microbiome as evidenced by any of a number of adverse health conditions. The adverse health conditions can include, but are not to be limited to obesity, diarrhea, diabetes, constipation, inflammatory bowel disease, abdominal gas, loss of appetite, poor weight gain, bacterial infection, lethargy, physical and emotional discomfort, or any combinations thereof. By way of example, in some aspects the botanical compositions can be fed to an animal throughout the duration of an adverse health condition and until the adverse health condition is no longer causing symptoms or duress in the animal, and where the animal can then be switched back to a diet that lacks the botanical compositions.

In some aspects, the botanical compositions herein can be fed prophylactically to the animals to prevent an adverse health condition. In some aspects, the botanical compositions herein can be fed to the animals at a first dosage, and if the animals experience an adverse health condition the animal could be switched to a diet including a botanical composition at a second dosage, where the second dosage is higher than the first dosage. Once an animal overcomes the adverse health condition consuming a diet at a second dosage, the animal can be returned to a diet at a first dosage or a diet having no botanical composition included therein. The first dosage and second dosage can include any dosage or a range of dosages in an amount effective to produce a beneficial effect, as described elsewhere herein. Further, in various aspects, once an animal overcomes the adverse health condition the animal can continue to consume the botanical compositions at either a first dosage or second dosage to maintain health as an every day dietary supplement.

The methods herein can include feeding animals a botanical composition directly, or the methods can include feeding animals a food product containing the botanical composition such that the administration of the botanical composition produces a beneficial effect on a gut health. In an aspect, the amount of botanical composition administered can be determined based on the concentration of botanical extract required to provide an amount effective to produce a beneficial effect on a gut health of an animal. The methods for using the botanical compositions herein can provide certain advantages to animals as compared to corresponding methods using food products that do not include the botanical compositions herein. The methods using the botanical compositions herein can produce a beneficial effect on a gut health in animals fed the botanical composition as observed relative to animals fed a diet lacking the botanical composition. In various aspects, the animals fed the botanical compositions herein include feline animals.

As described, the beneficial effects can be determined by comparing the effects observed in an animal administered a diet containing the botanical composition as compared to an animal fed a diet lacking the botanical composition. In some aspects, comparing the beneficial effects observed in an animal administered a diet containing the botanical composition can be compared to a different point in time when that same animal is fed a diet lacking the botanical composition. In some aspects, the different point in time can include a time when the animal is experiencing an adverse health condition and not consuming the botanical composition. In other aspects, comparing the beneficial effects observed in an animal administered a diet containing the botanical composition can be compared to an average value for a representative population of felines afflicted with one or more adverse health conditions. In various aspects, the beneficial effect is determined by comparing the effects observed in an animal administered a diet containing the botanical composition as compared to a different period of time when the animal was administered a diet lacking the botanical composition.

In various aspects, the methods herein include administering to an animal a botanical composition including one or more botanical extracts an amount effective to produce at least one health benefit to an animal, where the animal has one or more adverse health conditions including diarrhea, obesity, diabetes, inflammatory bowel disease, primary gastrointestinal disease, and severe gastrointestinal disease. In various aspects, the at least one health benefit includes cessation of diarrhea, a decrease in obesity, a reduction in diabetic biomarkers, an improvement of irritable bowel disease, or a decrease in incidence of primary gastrointestinal disease. In various aspects, the adverse health conditions can include one or more of diarrhea, obesity, diabetes, inflammatory bowel disease, primary gastrointestinal disease, and severe gastrointestinal disease. The methods herein further can include reducing one or more symptoms or intestinal distress in an animal, the symptoms including acute diarrhea, chronic diarrhea, constipation, and excessive vomiting.

The methods herein can include administering a botanical composition including one or more botanical extracts including one or more of an extract of dandelion leaf, dandelion leaf, ground dandelion leaf, dandelion root, Japanese ginseng, asafoetida, privit, common fennel, Indian mulberry, Rangoon creeper, long pepper, Indian lotus, cayenne/chili pepper, velvet foot winter mushroom, sugar apple, lion's mane mushroom, coriander, saw palmetto, Spanish dagger, mound lily yucca, carob, field lupine, goat's head, sweet prickly pear, partridge berry, bugleweed, Stelmatrocryption khasianum, Amaranthus spinosus, and elderberry, or combinations thereof.

The methods herein can include administering a botanical composition containing a botanical extract at an amount effective to produce a beneficial effect on the gut health of an animal from 0.0001 wt. % to 10 wt. %, or from 0.01 wt. % to 5.0 wt. %, or from 0.1 wt. % to 1.0 wt. % of a diet of an animal. The amount of one or more botanical extract effective to produce a beneficial effect on the gut health of an animal can be from 0.0001 wt. %, 0.001 wt. %, 0.01 wt. %, 0.1 wt. %, 0.2 wt. %, 0.3 wt. %, 0.4 wt. %, 0.5 wt. %, 0.6 wt. %, 0.7 wt. %, 0.8 wt. %, 0.9 wt. %, 1.0 wt. %, 1.25 wt. %, 1.5 wt. %, 1.75 wt. %, 2.0 wt. %, 2.25 wt. %, 2.5 wt. %, 2.75 wt. %, 3.0 wt. %, 3.25 wt. %, 3.5 wt. %, 3.75 wt. %, 4.0 wt. %, 4.25 wt. %, 4.5 wt. %, 4.75 wt. %, 5.0 wt. %, 6.0 wt. %, 7.0 wt. %, 8.0 wt. %, 9.0 wt. %, or 10 wt. % of the diet, or can be any amount falling within a range of any of the forgoing.

### Felines

The botanical compositions described herein can be formulated for any suitable domesticated cat at any suitable life stage. The term “feline” as used herein refers to domestic cats belonging to the genus and species, Felis catus or other non-domestic felines of the family Felidae. Domestic cats suitable for ingesting the botanical compositions herein can include, but are not to be limited to, breeds including Abyssinian, American Bobtail, American Curl, American Shorthair, American Wirehair, Balinese, Bengal, British Shorthair, Birman, Bombay, Burmese, Burmilla, Calico, Canadian Sphinx, Chartreux, Chausie, Cornish Rex, Cymric, Devon Rex, Domestic Long Hair, Domestic Medium Hair, Domestic Shorthair, Elf, Egyptian Mau, Exotic Shorthair, Havana, Himalayan, Japanese Bobtail, Javanese, Korat, Kurilian Bobtail, LaPerm, Lykoi, Maine Coon, Manx, Munchkin, Nebelung, Norwegian, Forest, Ocicat, Oriental Short Hair, Persian, Pixie-bob, Ragamuffin, Ragdoll, Russian Blue, Savannah, Scottish Fold, Selkirk Rex, Siamese, Siberian, Singapura, Snowshoe, Somali, Sphynx, Tabby, Tonkinese, Tortoiseshell, Toyger, Turkish Angora, Turkish Van, Tuxedo, York Chocolate, or any mixed breeds thereof. It will be appreciated that the botanical compositions herein further can be formulated for one or more non-domestic cats, including one or more wild species of cats such as those belonging to the genera Panthera, Lynx, Leptailurus, Leopardus, and Acinonyx.

### EXPERIMENTAL EXAMPLES

Various aspects of the present disclosure can be better understood by reference to the following Examples which are offered by way of illustration. The present disclosure is not limited to the Examples given herein.

### Example 1: Botanical Extracts

The botanical extracts analyzed herein include extract of dandelion leaf, dandelion leaf, ground dandelion leaf, dandelion root, Japanese ginseng, asafoetida (devil's dung), privit, common fennel, Indian mulberry, Rangoon creeper, long pepper, Indian lotus, cayenne/chili pepper, velvet foot winter mushroom, sugar apple, lion's mane mushroom, coriander, saw palmetto, Spanish dagger mound lily yucca, carob, field lupine, goat's head, sweet prickly pear, partridge berry, bugleweed, Stelmatrocryption khasianum (Sheng Teng), Amaranthus spinosus (spiny amaranth), and elderberry. All botanicals were extracted using dimethyl sulfoxide (DMSO) as described below.

### Example 2: Botanical Extract Modulation of Short-Chain Volatile Fatty Acid Profiles

The assay described in this example analyzed the volatile fatty acid production in representative samples of feline feces in an in vitro model of digestion.

Fresh feces from a single cat were collected and 30 g of a representative sample was immediately placed into the internal section of a filtered Seward Stomacher® bag. The fecal sample was transported to an anaerobic chamber (90% nitrogen, 5% carbon dioxide, 5% hydrogen) and 220 ml pre-reduced 0.1 M phosphate buffer (pH 6.8) was added, the bag was sealed, and the sample was homogenized for 2 minutes. A 10 ml volume of the filtered homogenate was added to 190 ml of a buffered salts media (8.0 mM NaCl, 0.7 mM Na₂SO₄, 50 mM KH₂PO₄, 0.654 mM CaCl₂₋₂H2O, 1.0 mM MgSO₄-7H₂O, resazurin (1 ml/L of 1 wt. % solution), 2.8 mM cysteine HCL-H₂O, 7.3 mM NaHCO₃, urea (10 ml/L 4 wt. % pre-reduced solution), and 14 mM FeSO₄-7H₂O, 1.6 mM ZnSO₄, 11 mM MnSO₄—H₂O, 0.50 mM CoCl₂-6H₂O, 11.1 mM CuCl₂—H₂O, Na₂MoO₄-2H₂O), mixed thoroughly, and distributed into 1.2 ml aliquots added to 10 replicate gas-tight 2 ml tubes containing pre-weighed food substrate and liquid (dry matter (DM) equivalent weight per volume (% wt./vol.)). Sample tubes were then placed on a tube rotator and allowed to ferment at 39° C. for 24 hours. Following fermentation, samples were analyzed for short-chain volatile fatty acids (VFA) by gas chromatography (Agilent DB FFAP, Santa Clara, CA). Exemplary VFAs measured herein include acetate, propionate, isobutyrate, butyrate, and isovalerate, and valerate. It will be appreciated that the VFAs measured herein are reported as the fatty acid anionic form, or conjugate base, of each respective acid. By way of example, acetate is the conjugate base of acetic acid.

Predigested cat food diet (a commercially available, inulin and supplement free cat food) was used as the food substrate. To simulate feline digestion, the diet was digested with 20 ml pepsin stock solution per 20 g predigested cat diet at pH 2.0, followed by 20 ml pancreatin stock solution per 20 g predigested cat food diet at pH 7.0. The pepsin stock solution was prepared by adding 7.5 g pepsin (Item P-7000, Sigma-Aldrich, St. Louis, MO, USA) per 150 ml double deionized water (ddH₂O). Pancreatin stock solution (Item P-1750, Sigma-Aldrich, St. Louis, MO, USA) was prepared by adding 9 g pancreatin to 90 mL ddH₂O, mixed, centrifuged for 5 minutes at 4,000 rpm.

The solids left after digestion were collected by filtration, washed, dried at 60° C. and added to each replicate tube at 12 mg (on a dry matter (DM) basis) to simulate diet residue in the hindgut. The test samples were non-digested and added to tubes in 6 mg (on a DM basis) quantities and repeated twice. The experimental test samples included the following botanical: extract of dandelion leaf, dandelion leaf, ground dandelion leaf, dandelion root, Japanese ginseng, asafoetida (devil's dung), privit, common fennel, Indian mulberry, Rangoon creeper, long pepper, Indian lotus, cayenne/chili pepper, velvet foot winter mushroom, sugar apple, lion's mane mushroom, coriander, saw palmetto, Spanish dagger mound lily yucca, carob, field lupine, goat's head, sweet prickly pear, partridge berry, bugleweed, Stelmatrocryption khasianum, Amaranthus spinosus, and elderberry. Each experimental test batch contained two controls including, a no botanical extract blank control and an inulin sample. Experimental test batches A and B were run according to the experimental design reported in Table 1.

TABLE 1
Test Batch Experimental Design

| Test Batch | Experimental Samples |
|---|---|
| A. | no botanical extract control |
| | inulin |
| | Dandelion leaf |
| | Velvet Foot Winter Mushroom |
| | Sugar Apple |
| B. | no botanical extract control |
| | inulin |
| | Rangoon Creeper |
| | Goats Head |
| | Sweet Prickly Pear |
| | Partridge Berry |

The VFAs were measured on a Perkin Elmer model Clarus 680 gas chromatograph and analyzed using Empower software. The effect of each botanical extract on intestinal microbial fermentation was determined by comparing differences in VFA production using the no botanical extract control as a negative control. Data was analyzed by one-way ANOVA (JMP 15 SAS Institute Inc.). The means for each test condition were compared across test groups using the Tukey-Kramer Test. Means that differed at P≤0.05 were determined to be statistically significant. The concentration of various VFAs including acetate, propionate, isobutyrate, butyrate, and isovalerate, valerate, and total VFAs were measured. The pH was also measured. Samples were run in quadruplicate or quintuplicate. Results of the analysis for each condition are presented in Table 2 and Table 3 as concentration of short-chain volatile fatty acid in mM with the standard deviation for each condition.

TABLE 2
Test Batch A: Effects of Botanicals on the Concentration of Short-
chain Volatile Fatty Acids Produced by the Feline Microbiome

| | no botanical | | | velvet foot | |
|---|---|---|---|---|---|
| | extract | | dandelion | winter | |
| VFA | control | inulin | leaf | mushroom | sugar apple |
| Acetate | 12.54 ± 0.98 | 33.39 ± 0.97 | 24.86 ± 0.99 | 25.24 ± 0.58 | 25.29 ± 1.00 |
| (mM) | | | | | |
| Propionate | 4.73 ± 0.09 | 13.72 ± 0.83 | 9.33 ± 0.27 | 21.96 ± 0.68 | 23.32 ± 0.87 |
| (mM) | | | | | |
| Isobutyrate | 0.52 ± 0.04 | 0.48 ± 0.13 | 1.64 ± 0.09 | 0.57 ± 0.18 | 0.58 ± 0.09 |
| (mM) | | | | | |
| Butyrate | 1.80 ± 0.07 | 8.32 ± 0.39 | 3.22 ± 0.11 | 7.42 ± 0.29 | 7.31 ± 0.19 |
| (mM) | | | | | |
| Isovalerate | 1.07 ± 0.08 | 0.35 ± 0.03 | 2.58 ± 0.13 | 0.65 ± 0.03 | 0.51 ± 0.04 |
| (mM) | | | | | |
| Valerate | 0.38 ± 0.19 | 1.29 ± 0.04 | 1.93 ± 0.05 | 1.18 ± 0.05 | 1.10 ± 0.04 |
| (mM) | | | | | |
| Total VFA | 21.04 ± 1.3 | 57.54 ± 1.67 | 43.56 ± 1.40 | 57.02 ± 1.43 | 58.10 ± 2.09 |
| (mM) | | | | | |
| pH | 7.4 ± 0.02 | 6.75 ± 0.03 | 7.11 ± 0.02 | 6.76 ± 0.01 | 6.71 ± 0.02 |

TABLE 3
Test Batch B: Effects of Botanicals on the Concentration of Short-
chain Volatile Fatty Acids Produced by the Feline Microbiome

| | no botanical | | Rangoon | | sweet prickly | partridge |
|---|---|---|---|---|---|---|
| VFA | extract control | inulin | creeper | goats head | pear | berry |
| Acetate | 9.61 ± 0.99 | 33.89 ± 2.54 | 16.09 ± 1.10 | 18.57 ± 0.75 | 22.34 ± 0.93 | 12.60 ± 0.59 |
| (mM) | | | | | | |
| Propionate | 3.28 ± 0.26 | 20.78 ± 0.70 | 7.31 ± 0.33 | 8.44 ± 0.25 | 7.59 ± 0.16 | 9.10 ± 0.33 |
| (mM) | | | | | | |
| Isobutyrate | 0.64 ± 0.20 | 1.00 ± 0.13 | 0.99 ± 0.23 | 1.34 ± 0.41 | 1.91 ± 0.58 | 0.73 ± 0.27 |
| (mM) | | | | | | |
| Butyrate | 0.54 ± 0.21 | 2.84 ± 0.22 | 2.34 ± 0.37 | 2.41 ± 0.14 | 3.41 ± 0.21 | 0.43 ± 0.11 |
| (mM) | | | | | | |
| Isovalerate | 1.16 ± 0.26 | 0.46 ± 0.10 | 1.53 ± 0.10 | 1.73 ± 0.12 | 2.44 ± 0.16 | 0.26 ± 0.09 |
| (mM) | | | | | | |
| Valerate | 0.08 ± 0.06 | 0.12 ± 0.06 | 0.14 ± 0.05 | 0.13 ± 0.04 | 1.08 ± 0.06 | 0.09 ± 0.04 |
| (mM) | | | | | | |
| Total VFA | 15.32 ± 1.82 | 59.09 ± 3.19 | 28.41 ± 1.71 | 32.62 ± 0.96 | 38.78 ± 0.99 | 23.21 ± 0.84 |
| (mM) | | | | | | |
| pH | 7.55 ± 0.01 | 6.97 ± 0.43 | 7.33 ± 0.02 | 7.32 ± 0.01 | 7.23 ± 0.02 | 7.28 ± 0.02 |

Results indicate that sugar apple, followed by velvet foot winter mushroom produced a higher amount of propionate as compared to inulin. For butyrate, sugar apple, followed by velvet food winter mushroom produced the highest levels of butyrate. Dandelion leaf produced the highest levels of acetate. Sugar apple, followed by velvet food winter mushroom produced the highest levels of total VFAs of all botanicals assayed.

### Example 3: Effects of Botanical Extracts on the Metagenomic Profile of the Feline Microbiome In Vitro

Cat fecal samples were analyzed to assess changes in the microbiome in response to each botanical extract as outlined in Example 2.

Genomic DNA was extracted using the ZymoBIOMICS 96 MagBead DNA kit equipped with a Biomek i7 automated liquid handler (Beckman Coulter Life Sciences, Indianapolis, IN). Nanopore sequencing libraries were performed using the Rapid PCR-Barcoding kit (Oxford Nanopore Technologies, Oxford, UK). Samples were multiplexed at 12 samples per flow cell and sequenced for 72 hours.

Taxonomic assignation was performed using the Kraken2 taxonomic sequence classification system (Bayesian Reestimation of Abundance with Kraken, Johns Hopkins University Center for Computational Biology, Baltimore, MD) with a curated database containing publicly available genomes from RefSeq (https://www.ncbi.nlm.nih.gov/refseq/) and metagenomics studies. Estimation of abundance at the species level was done with Bracken (Bayesian Reestimation of Abundance with Kraken, John's Hopkins University Center for Computational Biology, Baltimore, MD). Counts were normalized using Center Log Ratio (CLR) with the R package, zCompositions, to impute zeros.

Without being bound by any particular theory, it is understood that the data obtained by nanopore sequencing is compositional in nature, such that the abundance of any given nucleotide fragment of a representative taxon present in a given sample is only interpretable relative to another different nucleotide fragment from a different representative taxon in the same sample. The amount of any given nucleotide in a sample can be an artifact of the sequencing method and thus can change from sample to sample. However, it is still possible to determine the relative abundances of taxa across different samples using these techniques. After taxonomic assignation as described above, read counts are assigned to all taxa within a given sample and ratios of the read counts between the different taxa can be determined in order to make comparisons within the data sets. As such, the data must be transformed such that relative comparisons can be made. In various aspects the transformation can be interpreted as a normalization. Centered Log-Ratio (CLR) transformation values can be calculated by taking the log of the ratio of the taxon read count within a sample to the geometric mean of the read counts of all taxa within the sample. Since the CLR values for each sample are log transformed due to the nature of this transformation, when comparing these values across samples the numerical differences are equivalent to a log fold change. By way of example, when comparing two samples A and B for a species X, (i.e., when looking at X_{clrB}-X_{clrA}, a value of 0 for the difference between two samples refers to the same log ratio and no change between the two samples. A positive value indicates that the relative abundance is higher in sample B as compared to sample A and the log fold difference reported as a positive value. Similarly, a negative value indicates that the relative abundance is higher in sample A as compared to sample B. Methods for determining the centered log-ratio are described, for example, in Gloor et al., “Microbiome Datasets Are Compositional: And This Is Not Optional,” Frontiers in Microbiology, 2017, vol. 8, article 2224. https://doi.org/10.3389% 2Ffmicb.2017.02224.

Ecological diversity analysis was performed using the R packages Phyloseq and Vegan were used to perform alpha diversity analysis. For the alpha diversity analysis, library size differences were adjusted first using the rarefaction method.

Overall characterization of the microbial community at the species level (raw counts and center-log ratio transformed values) and the derived measures (such as Shannon diversity) were determined for the microbial community. Centered log-ratio (CLR) values of the species of interest (e.g., Actinobacteria, Bacteroidaceae, Bifidobacterium, Collinsella, Enterobacteriaceae, Enterococcus faecalis, Escherichia coli, Faecalibacterium, Peptostreptococcaceae, Prevotella, Roseburia, Ruminococcaceae, Bacteroidetes, Salmonella, Lactobacillus, and Enterococcus hirae), Shannon Diversity values and standard deviations are summarized in Table 4 and Table 5.

TABLE 4
Experimental Batch A: Effects of Botanical Extracts on the Relative
Abundance of Bacterial Species of Interest in the Feline Microbiome

| | | | velvet foot | | |
|---|---|---|---|---|---|
| | | | winter | sugar | dandelion |
| Name | Blank | inulin | mushroom | apple | leaf |
| Actinobacteria | 3.370 | 2.302 | 2.278 | 3.016 | 3.310 |
| | (±0.420) | (±0.143) | (±0.177) | (±0.210) | (±0.183) |
| Bacteroidaceae | 6.229 | 6.651 | 6.689 | 7.230 | 7.519 |
| | (±0.096) | (±0.149) | (±0.123) | (±0.119) | (±0.080) |
| Bifidobacterium | 4.015 | 2.993 | 2.820 | 3.233 | 4.404 |
| | (±0.148) | (±0.127) | (±0.091) | (±0.140) | (±0.195) |
| Collinsella | 5.378 | 5.727 | 4.676 | 4.743 | 5.164 |
| | (±0.173) | (±0.138) | (±0.063) | (±0.146) | (±0.139) |
| Enterobacteriaceae | 8.076 | 7.264 | 7.667 | 7.286 | 6.348 |
| | (±0.284) | (±0.324) | (±0.126) | (±0.114) | (±0.114) |
| Enterococcus faecalis | 2.562 | 2.399 | 4.438 | 3.276 | 1.193 |
| | (±0.422) | (±0.323) | (±0.100) | (±0.351) | (±1.985) |
| Escherichia coli | 10.334 | 9.521 | 9.993 | 9.290 | 8.433 |
| | (±0.247) | (±0.376) | (±0.066) | (±0.144) | (±0.109) |
| Faecalibacterium | −3.681 | 2.838 | 3.471 | 3.014 | 3.847 |
| | (±0.461) | (±0.133) | (±0.090) | (±0.226) | (±0.145) |
| Peptostreptococcaceae | 5.635 | 3.694 | 4.250 | 3.879 | 5.238 |
| | (±0.390) | (±0.199) | (±0.124) | (±0.188) | (±0.194) |
| Prevotella | 7.392 | 6.691 | 6.986 | 7.098 | 7.099 |
| | (±0.212) | (±0.125) | (±0.081) | (±0.167) | (±0.076) |
| Roseburia | 0.651 | 0.524 | −0.096 | 1.724 | 1.801 |
| | (±1.997) | (±1.802) | (±2.339) | (±0.242) | (±0.169) |
| Ruminococcaceae | 5.205 | 4.202 | 4.175 | 3.663 | 5.198 |
| | (±0.200) | (±0.194) | (±0.105) | (±0.161) | (±0.111) |
| Bacteroidetes | 5.822 | 4.557 | 5.697 | 6.494 | 6.626 |
| | (±0.438) | (±0.143) | (±0.079) | (±0.146) | (±0.063) |
| Salmonella | 0.000 | 0.000 | 0.000 | 0.000 | 0.000 |
| Lactobacillus | 0.000 | 0.000 | 0.000 | 0.000 | 0.000 |
| Enterococcus hirae | 0.000 | 0.000 | 0.000 | 0.000 | 0.000 |
| Shannon Diversity | 2.526 | 3.128 | 1.996 | 2.749 | 3.392 |
| Index | (±0.163) | (±0.144) | (±0.094) | (±0.073) | (±0.071) |

TABLE 5
Experimental Batch B: Effects of Botanical Extracts on the Relative
Abundance of Bacterial Species of Interest in the Feline Microbiome

| | | | | | sweet | |
|---|---|---|---|---|---|---|
| | | | Rangoon | goat's | prickly | partridge |
| Name | Blank | inulin | creeper | beard | pear | berry |
| Actinobacteria | 0.737 | −0.056 | 0.764 | 0.489 | 1.226 | 0.162 |
| | (±0.363) | (±0.229) | (±0.353) | (±0.113) | (±0.135) | (±0.360) |
| Bacteroidaceae | 5.956 | 6.475 | 6.175 | 6.122 | 7.426 | 7.652 |
| | (±0.152) | (±0.294) | (±0.263) | (±0.118) | (±0.065) | (±0.460) |
| Bifidobacterium | 3.258 | 2.514 | 2.824 | 2.319 | 2.693 | 3.611 |
| | (±0.104) | (±0.140) | (±0.175) | (±0.261) | (±0.062) | (±0.223) |
| Collinsella | 4.213 | 3.485 | 3.737 | 3.467 | 3.568 | 4.230 |
| | (±0.200) | (±0.176) | (±0.161) | (±0.251) | (±0.084) | (±0.237) |
| Enterobacteriaceae | 7.664 | 8.474 | 7.012 | 7.716 | 5.138 | 7.832 |
| | (±0.185) | (±0.291) | (±0.188) | (±0.129) | (±0.066) | (±0.222) |
| Enterococcus | 4.397 | −2.309 | −2.917 | −2.958 | −3.251 | 4.976 |
| faecalis | (±0.647) | (±0.344) | (±0.215) | (±0.394) | (±0.301) | (±0.213) |
| Escherichia coli | 10.368 | 10.875 | 9.312 | 9.928 | 7.284 | 10.493 |
| | (±0.167) | (±0.373) | (±0.308) | (±0.292) | (±0.242) | (±0.145) |
| Faecalibacterium | −3.878 | −2.447 | −3.235 | −4.088 | −2.749 | −2.905 |
| | (±0.438) | (±1.773) | (±1.581) | (±0.442) | (±2.262) | (±0.484) |
| Peptostreptococcaceae | 5.482 | 3.748 | 4.894 | 4.995 | 3.881 | 2.457 |
| | (±0.182) | (±0.483) | (±0.116) | (±0.145) | (±0.131) | (±0.940) |
| Prevotella | 4.844 | 5.725 | 4.883 | 3.615 | 3.204 | 4.084 |
| | (±0.160) | (±0.187) | (±0.266) | (±0.218) | (±0.130) | (±0.253) |
| Roseburia | 0.811 | −3.307 | −0.432 | −1.178 | 1.477 | −2.905 |
| | (±0.173) | (±0.306) | (±1.913) | (±2.328) | (±0.143) | (±0.484) |
| Ruminococcaceae | 3.636 | 2.301 | 3.707 | 2.586 | 3.441 | 1.921 |
| | (±0.296) | (±0.298) | (±0.308) | (±0.159) | (±0.053) | (±0.780) |
| Bacteroidetes | 3.423 | 4.055 | 4.006 | 3.904 | 5.529 | 4.956 |
| | (±0.377) | (±0.262) | (±0.427) | (±0.087) | (±0.042) | (±0.354) |
| Salmonella | −0.453 | 0.994 | −4.059 | 0.203 | −4.444 | −0.978 |
| | (±2.828) | (±2.327) | (±0.297) | (±2.134) | (±0.163) | (±2.192) |
| Lactobacillus | 0.000 | 0.000 | 0.000 | 0.000 | 0.000 | 0.000 |
| Enterococcus hirae | −2.415 | −2.309 | −2.917 | −2.958 | −3.251 | −0.928 |
| | (±0.425) | (±0.344) | (±0.215) | (±0.394) | (±0.301) | (±2.778) |
| Shannon Diversity | 1.787 | 1.562 | 2.631 | 2.250 | 2.647 | 2.130 |
| Index | (±0.096) | (±0.256) | (±0.164) | (±0.106) | (±0.089) | (±0.059) |

Results indicate that dandelion leaf followed by partridge berry led to strongest increase in Bifidobacterium levels. Sugar apple, dandelion leaf, sweet prickly pear, partridge berry and velvet foot winter mushroom led to strongest increase in Bacteroidaceae levels. Dandelion leaf led to the strongest increase in Ruminococcaceae levels. Dandelion leaf followed by velvet foot winter mushroom, and sugar apple led to the strongest increase in Faecalibacterium levels.

### Example 4: Effects of Botanical Extracts on Bacterial Growth

The assay described in this example includes an in vitro activation assay to monitor the effect that the experimental botanical extracts had on growth characteristics of various beneficial intestinal bacteria. Each beneficial bacterial strain was cultured on agar plates according to the growth conditions for each respective bacterial strain as presented in Table 6.

The activation assay was performed in 96-well plates by inoculating the corresponding culture media at an initial OD₆₀₀ (i.e., optical density at 600 nm). Briefly, 145 μl of assay medium, 145 μl of a respective bacterial strain slurry, and 5 μl of each respective botanical extract were added to respective wells of the 96-well plate. The botanical extracts were tested along with a baseline no botanical extract control, which included the respective preferred media plus water or DMSO to simulate the solvent used for making the botanical stocks, and a positive control, which included the respective preferred media plus MRS, which is known to stimulate growth. Each sample was tested in triplicate.

Botanical extracts were tested at 0.6 g/L in DMSO. Stocks were generated by dissolving each botanical stock separately in DMSO and shaking at 250 revolutions per minute (rpm) for 30 minutes. Stocks were removed from the shaker and left to sit for 15 mins at room temperature to allow insoluble sample matter to settle to the bottom. The supernatants were removed and stored at −20° C. until future use.

Activation media was chosen based on the modified standard ileal efflux medium (SIEM) composition according to Minekus et al., 1999, and modified in (g/L) and shown in Table 7. Yeast casitone fatty acids broth with carbohydrates (YCFAC Broth) and membrane tryptone glucose extract anaerobic enrichment broth (MTGE Broth) were purchased from Anaerobe Systems, Inc. (Morgan Hills, CA, USA).

The 96-well plates were incubated for 48 hours at 37° C. with constant shaking using the Biotek LogPhase 600 instrument from Agilent (Agilent Technologies, Inc., Santa Clara, CA). The OD₆₀₀ was measured every 20 minutes and recorded. A Python script was created and used to analyze growth data to determine lag time, growth rate, and total growth for each bacterial culture for each condition tested.

TABLE 6
Activation Assay Experimental Conditions

| | | | Agar Plate | Assay | Starting |
|---|---|---|---|---|---|
| Strain Name | Source | Culture Media | Media | Conditions | OD₆₀₀ |
| Lactobacillus | DSM¹ | Activation media | MRS³ | 37 °C, | 0.02 |
| reuteri | #20016 | | | Aerobic | |
| Enterococcus | Isolated from | Activation media | MRS³ | 37 °C, | 0.02 |
| hirae | domestic cat. | | | Aerobic | |
| Bifidobacterium | Proctor and | Activation media | MRS³ | 37 °C, | 0.05 |
| longum | Gamble | | | Anaerobic | |
| Roseburia | DSM | 70% YCFAC⁴ | BRU⁵ | 37 °C, | 0.05 |
| inulinivarons | #102148 | broth in ddH₂O | | Anaerobic | |
| Ruminococcaceae | ATCC ®² | 70% MTGE⁶ | YCFAC⁴ | 37 °C, | 0.05 |
| | #TSD-27 | broth in ddH₂O | | Anaerobic | |

¹Deutsche Sammlung von Mikroorganismen und Zellkulturen GmbH - i.e., German Collection of Microorganisms and Cell Cultures GmbH; Braunschweig, Germany
²American Type Culture Collection; Manassas, Virginia
³De Man, Rogosa and Sharpe agar
⁴yeast casitone fatty acids agar with carbohydrates
⁵Brucella blood agar
⁶membrane tryptone glucose extract anaerobic enrichment

TABLE 7
Activation Media Used in the Activation Assays

| Reagent | Source | g/L | pH |
|---|---|---|---|
| pectin from citrus | Cargill, Inc. | 0.047 | 5.8 |
| xylan from beechwood | Sigma Aldrich, Inc. | 0.047 | 5.8 |
| arabinogalactan from larch wood | Sigma Aldrich, Inc. | 0.047 | 5.8 |
| amylopectin from maize | Sigma Aldrich, Inc. | 0.047 | 5.8 |
| starch from potato | Sigma Aldrich, Inc. | 0.392 | 5.8 |
| pancreatic digest of casein | Bio-world Molecular Life Sciences | 24.0 | 5.8 |
| Tween 80 | Thermo Fischer Scientific | 17.0 | 5.8 |
| Oxiod ™ Proteose Peptone | Thermo Fischer Scientific | 24.0 | 5.8 |
| bovine bile | Sigma Aldrich, Inc. | 0.4 | 5.8 |
| L-Cysteine Hydrochloride | Thermo Fischer Scientific | 0.2 | 5.8 |

The effect the botanical extracts had on the growth of various bacterial strains was assayed and compared to control growth conditions with no botanical extracts present. An internal activation assay scoring system was used to determine the effect of each ingredient on growth rate, lag time, and change in OD₆₀₀. If significant changes (i.e., p<0.05, t-test) were identified compared with baseline control, the activation assay score was calculated as the effect size ×3 (effect size times three). If there were significant changes compared to the positive control, the activation assay score was the effect size ×6 (effect size times six). A final activation assay score was calculated as the cumulative activation assay score across growth rate, lag time, and change in OD₆₀₀. It will be appreciated that as the activation assay score increases in the positive direction, the botanical extract has a stronger effect on promoting bacterial growth while as the activation assay score increases in the negative direction the botanical extract has a stronger effect on inhibiting bacterial growth. A score of zero is indicative of no statistically significant effect of the botanical extract on the growth of the bacterial strain. Overall cumulative activation assay scores for each botanical extract tested, taking the three factors into account, i.e., growth rate, lag, and change in OD₆₀₀ are tabulated in Table 8.

TABLE 8
Cumulative Activation Assay Scores for Botanical Extracts

| Botanicals | | | | | Ruminococcaceae |
|---|---|---|---|---|---|
| Tested | L. reuteri | E. hirae | R. inulinivarons | B. longum | sp. |
| dandelion leaf | 3.22 | 4.02 | 0.00 | −0.86 | 4.25 |
| Japanese ginseng | 3.70 | 3.67 | 0.00 | 3.45 | 3.31 |
| Japanese ginseng | 0.00 | 7.03 | −3.62 | −0.83 | 4.43 |
| asafoetida | 0.00 | 0.00 | 0.00 | −0.86 | 1.17 |
| privit | 8.70 | 7.50 | −3.71 | 7.40 | −0.84 |
| common fennel | −3.51 | 3.58 | 0.00 | 0.22 | 1.17 |
| Indian mulberry | −3.34 | 0.00 | 0.00 | −0.86 | 0.00 |
| Rangoon creeper | −0.91 | −1.04 | −0.39 | 0.00 | −3.87 |
| long pepper | −0.94 | 0.20 | 0.00 | 0.22 | 0.00 |
| Indian lotus | −4.07 | −4.91 | 0.00 | −8.33 | 1.17 |
| cayenne/chili | −6.59 | 0.00 | 0.00 | 0.00 | −5.58 |
| pepper | | | | | |
| velvet foot, | 0.00 | 7.57 | 0.00 | 2.93 | 6.79 |
| winter mushroom | | | | | |
| sugar apple | 8.38 | 8.33 | 0.00 | 7.94 | 7.21 |
| lion's mane | 3.48 | 8.30 | 0.00 | −0.77 | 5.97 |
| mushroom | | | | | |
| coriander | 0.00 | 0.00 | 0.00 | 0.22 | 1.17 |
| saw palmetto | 0.00 | 7.00 | 0.00 | 2.84 | 0.00 |
| Spanish dagger | −0.94 | 0.00 | 7.25 | 0.00 | 1.14 |
| mound lily yucca | | | | | |
| carob | 0.00 | 0.15 | 0.00 | −3.62 | 3.41 |
| field lupine | −1.39 | −5.18 | 0.00 | −3.95 | −14.24 |
| goat's head | 0.00 | 0.00 | 0.00 | −3.40 | 1.20 |
| sweet prickly | 0.00 | 3.40 | 3.03 | 0.00 | 1.16 |
| pear | | | | | |
| partridge berry | 0.00 | −4.17 | 0.00 | 0.00 | 1.24 |
| bugleweed | 3.79 | 6.55 | −5.86 | 0.00 | 3.22 |
| Stelmatrocryption | 0.00 | 2.28 | 0.00 | 0.00 | 3.18 |
| khasianum | | | | | |
| Amaranthus | 0.00 | 0.00 | 0.00 | 0.00 | 1.20 |
| spinosus | | | | | |
| elderberry | 7.13 | 10.05 | 0.00 | 3.22 | 3.55 |

Results indicate that privit, followed by sugar apple and elderberry are the most potent activators of L. reuteri. Elderberry, followed by sugar apple, lion's mane mushroom, velvet foot winter mushroom, privit, Japanese ginseng are the most potent activators of E. hirae. Spanish dagger mound lily yucca, followed by sweet prickly pear the most potent activators of R. inulinivarons. Sugar apple followed by privit, Japanese ginseng and elderberry are the most potent activators of B. longum. Sugar apple, followed by velvet foot winter mushroom, and lion's mane mushroom, Japanese ginseng are the most potent activators of Ruminococcaceae sp.

### Example 5: Effects of Botanical Extracts on Bacterial Growth Inhibition

The assay described in this example was performed as an in vitro model to determine the minimum inhibitory concentration (MIC) for the botanical extracts tested against the various strains of pathogenic or detrimental bacteria. The MIC is the lowest concentration of the product that inhibits the growth of the bacterial organism tested.

Botanical extract stocks were prepared by dissolving 25 mg of each in 1 ml DMSO (dilution of 0.25% weight per volume (i.e., % wt./vol.) and extracting for 30 min with gentle agitation as discussed in Example 4. The botanical stocks were removed from the shaker and left to sit for 15 mins at room temperature to allow insoluble sample matter to settle to the bottom. The supernatants were removed and stored at −20° C. until future use.

Bacterial growth suppression in the presence of the botanical extracts was determined at eight different concentrations using 2-fold serial dilutions with a starting concentration of 0.125% (wt./vol. %) extracted botanical product diluted in the appropriate growth media for each microbe. Additional dilutions tested include, 0.0625% wt./vol., 0.03125% wt./vol., 0.015625% wt./vol., 0.0078% wt./vol., 0.0039% wt./vol., 0.0019% wt./vol., 0.00097% wt./vol. The dilution series were inoculated with broth-grown bacterial cultures to achieve a starting concentration of approximately 5× 105 colony forming units/ml (cfu/ml) and were incubated overnight. Sterility of the botanical extracts in each of the culture reagents was confirmed using the bacterial extracts and broth only controls for each preparation. A dilution series of DMSO was also evaluated for each bacterial species to rule out any detrimental effects of the solvent alone on the growth of the bacteria. The bacteria used, assay media, and growth conditions are outlined in Table 9.

TABLE 9
Bacterial Strains, Assay Media, and Growth Conditions for Inhibition Assays

| | | Assay | Temp. | |
|---|---|---|---|---|
| Strain Name | Source | Media | (°C) | Condition |
| Enterococcus faecalis | ATCC ® | Iso-Sensitest | 37 °C | Aerobic |
| | 2912 | Broth | | |
| Enterococcus faecalis | Field | Iso-Sensitest | 37 °C | Aerobic |
| | Isolate | Broth | | |
| Desulfovibrio vulgaris | ATCC ® | Brain Heart | 37 °C | Anaerobic |
| | 29579 | Infusion | | |
| | | Broth | | |
| Streptococcus canis | ATCC ® | Iso-Sensitest | 37 °C | Aerobic |
| | 43497 | Broth | | |
| Collinsella aerofaciens | ATCC ® | Brain Heart | 37 °C | Anaerobic |
| | 25986 | Infusion | | |
| | | Broth | | |
| Salmonella enterica | ATCC ® | Iso-Sensitest | 37 °C | Aerobic |
| subsp. enterica serovar | 14028 | Broth | | |
| Typhimurium | | | | |
| Escherichia coli | ATCC ® | Tryptic Soy | 37 °C | Aerobic |
| | 14028 | Agar | | |
| Campylobacter jejuni | ATCC ® | Brain Heart | 37 °C | Microaerophilic |
| | 33560 | Infusion | | CO₂: 7-10% |
| | | Broth | | O₂: 3-5% |
| Campylobacter | ATCC ® | Brain Heart | 37 °C | Microaerophilic |
| helveticus | 51209 | Infusion | | CO₂: 7-10% |
| | | Broth | | O₂: 3-5% |
| Burkholderia cepacia | ATCC ® | Iso-Sensitest | 30 °C | Aerobe |
| | 25416 | Broth | | |
| Clostridium perfringens | ATCC ® | Brain Heart | 37 °C | Anaerobe |
| | 13124 | Infusion | | |
| | | Broth | | |

Overall, the efficacy of the botanical extracts in inhibiting bacterial strain growth is summarized in Table 10.

TABLE 10
Inhibition Assay Scores for Botanical Extracts

| | Minimum | |
|---|---|---|
| | Inhibitory | |
| | Concentration | # Strains Effective |
| Name | (% wt./vol.) | Against |
| dandelion leaf | 0.0625 | 1 |
| privit | 0.125 | 1 |
| Rangoon creeper | 0.125 | 1 |
| saw palmetto | 0.125 | 1 |
| field lupine | 0.0625 | 1 |
| sweet prickly pear | 0.125 | 1 |
| Amaranthus spinosus | 0.125 | 1 |
| Stelmatrocryption | 0.0625 | 2 |
| khasianum | | |
| Indian mulberry | 0.03125 | 3 |
| coriander | 0.015625 | 3 |
| goat's head | 0.03125 | 3 |
| Indian lotus | 0.03125 | 4 |
| common fennel | 0.03125 | 5 |
| cayenne/chili pepper | 0.0078 | 6 |

The following botanical extracts showed a minimum inhibitory concentration (MIC) against one bacterial strain: dandelion leaf (MIC: 0.0625% wt./vol, for Clostridium perfringens); privit (MIC: 0.125% wt./vol, for Burkholderia cepacia); Rangoon creeper (MIC: 0.125% wt./vol. for Burkholderia cepacia); saw palmetto (MIC: 0.125% wt./vol, for Desulfovibrio vulgaris); field lupine (MIC: 0.0625% wt./vol, for Clostridium perfringens); sweet prickly pear (MIC: 0.125% wt./vol, for Desulfovibrio vulgaris); and Amaranthus spinosus (MIC: 0.125% wt./vol. for Burkholderia cepacia).

The following botanical extracts showed a MIC against two bacterial strains: Stelmatrocryption khasianum (MIC of 0.125% wt./vol, for Burkholderia cepacia and MIC: 0.0625% wt./vol, for Collinsella aerofaciens).

The following botanical extracts showed a MIC against three bacterial strains: Indian mulberry (MIC: 0.125% wt./vol, for Burkholderia cepacia, MIC: 0.125% wt./vol. for Streptococcus canis, and 0.03125% wt./vol, for Clostridium perfringens); coriander (MIC: 0.0625% wt./vol, for Burkholderia cepacia, MIC: 0.0625% wt./vol, for Streptococcus canis, and MIC: 0.015625% wt./vol, for Desulfovibrio vulgaris); and goat's head (MIC: 0.125% wt./vol. for Burkholderia cepacia, MIC: 0.0625% wt./vol, for Streptococcus canis, and MIC: 0.03125% wt./vol, for Desulfovibrio vulgaris).

The following botanical extracts showed a MIC against four bacterial strains: Indian lotus (MIC: 0.03125% wt./vol, for Burkholderia cepacia, MIC: 0.015625% wt./vol. for Campylobacter helveticus, MIC: 0.015625% wt./vol, for Campylobacter jejuni, and MIC: 0.00391% wt./vol, for Clostridium perfringens).

The following botanical extracts showed a MIC against five bacterial strains: common fennel (MIC: 0.125% wt./vol, for Burkholderia cepacia, MIC: 0.125% wt./vol, for Streptococcus canis, MIC: 0.125% wt./vol, for Collinsella aerofaciens, MIC: 0.03125% wt./vol. for Desulfovibrio vulgaris, and MIC: 0.03125% wt./vol, for Clostridium perfringens).

The following botanical extracts showed a MIC against six bacterial strains: cayenne/chili pepper (MIC: 0.0625% wt./vol, for Enterococcus faecalis, MIC: 0.125% wt./vol. for Burkholderia cepacia, MIC: 0.0156% wt./vol, for Streptococcus canis, MIC: 0.0625% wt./vol. for Collinsella aerofaciens, MIC: 0.00781% wt./vol, for Desulfovibrio vulgaris, and MIC: 0.0625% wt./vol, for Clostridium perfringens).

### Example 6: Effects of Botanical Extracts on Feline Gut Microbiome in a Digestion and Fermentation Model

This example assayed the effects of various botanical extracts on the feline gut microbiota using a feline digestion and fermentation model. The model simulated the feline gut microbiota using individually sealed bioreactors containing fecal inoculum and nutrients in sealed bioreactors containing a headspace volume and ports for sampling.

The experiments described in this example detail the effects of various botanical extracts on: (i) overall metabolic activity (pH and gas production); (ii) saccharolytic (lactate and short-chain fatty acids including acetate, propionate, butyrate, and valerate) and proteolytic (ammonium and branched-chain fatty acids including isobutyrate, isovalerate, and isocaproate; and total short chain fatty acids) metabolites; and (iii) bacterial community composition as assessed by Nanopore shotgun sequencing.

The digestion simulation model system includes a series of individually sealed, independent bioreactors each containing a headspace volume above a sample chamber containing feline fecal matter, sampling ports for monitoring gas production and pH, and temperature control to maintain the temperature of each bioreactor at 37° C. for each experimental condition. A total of 6 feline donors were assayed (n=6).

Three botanical extracts, including dandelion leaf, velvet foot winter mushroom, and sugar apple. Control samples including inulin and a non-botanical extract blank including medium and microbiome inoculum were also included. The botanical extracts were used at a concentration of 5 mg/ml. Control samples included inulin and a non-botanical extracts blank containing medium and microbiome inoculum. The concentrations of acetate, propionate, butyrate, valerate and branched chain fatty acids (BCFA) (i.e., the sum of isobutyrate, isovalerate and isocaproate) were determined via a gas chromatography with flame ionization detector (GC-FID) approach. The pH and gas production were also measured as key markers for microbial metabolic activity. Results of the fermentation parameters are presented in Table 11.

TABLE 11
Effects of Botanical Extracts on the Concentration of Short-chain
Volatile Fatty Acids Produced by the Feline Microbiome (n = 6)

| | | | | | | Velvet | |
|---|---|---|---|---|---|---|---|
| | | | | | | Foot | |
| | | | | | Dandelion | Winter | Sugar |
| | | Blank | Blank | Inulin | Leaf | Mushroom | Apple |
| | | 0 hours | 24 hours | 24 hours | 24 hours | 24 hours | 24 hours |
| pH | Mean | 6.49 | 6.52 | 5.86 | 6.38 | 6.01 | 6.21 |
| | Std | 0.03 | 0.03 | 0.04 | 0.05 | 0.05 | 0.07 |
| Gas | Mean | 0.00 | 148.98 | 501.80 | 219.45 | 407.29 | 305.02 |
| production | Std | 0.00 | 13.10 | 77.16 | 19.11 | 74.20 | 42.17 |
| (mbar) | | | | | | | |
| Acetate | Mean | 0.47 | 7.25 | 20.85 | 12.97 | 18.32 | 14.29 |
| | Std | 0.13 | 1.53 | 4.05 | 1.81 | 5.76 | 4.72 |
| Propionate | Mean | 0.22 | 3.73 | 11.84 | 5.98 | 12.35 | 7.50 |
| | Std | 0.10 | 0.89 | 2.45 | 1.10 | 3.74 | 2.52 |
| Butyrate | Mean | 0.12 | 2.36 | 5.12 | 4.26 | 6.15 | 3.65 |
| | Std | 0.04 | 0.74 | 2.34 | 1.18 | 2.31 | 1.36 |
| Valerate | Mean | 0.04 | 1.25 | 1.48 | 2.11 | 2.67 | 1.56 |
| | Std | 0.03 | 0.69 | 1.53 | 0.61 | 2.08 | 1.36 |
| Isobutyrate | Mean | 0.01 | 0.70 | 0.53 | 0.92 | 0.66 | 0.64 |
| | Std | 0.01 | 0.30 | 0.27 | 0.18 | 0.27 | 0.27 |
| Isovalerate | Mean | 0.03 | 1.44 | 0.83 | 1.76 | 1.19 | 1.23 |
| | Std | 0.01 | 0.55 | 0.43 | 0.20 | 0.46 | 0.49 |
| BCFA | Mean | 0.04 | 2.14 | 1.37 | 2.68 | 1.84 | 1.87 |
| | Std | 0.02 | 0.85 | 0.69 | 0.37 | 0.73 | 0.76 |
| total SCFA | Mean | 0.89 | 16.73 | 40.66 | 28.01 | 41.33 | 28.86 |
| | Std | 0.25 | 1.86 | 4.33 | 2.87 | 4.43 | 4.33 |

Cat fecal samples were analyzed to assess changes in the microbiome in response to each botanical extract. The experimental samples contained the botanical extracts including dandelion leaf, velvet foot winter mushroom, sugar apple, and controls included inulin and a non-botanical extract blank including medium and microbiome inoculum.

Genomic DNA was extracted using the ZymoBIOMICS 96 MagBead DNA kit equipped with a Biomek i7 automated liquid handler (Beckman Coulter Life Sciences, Indianapolis, IN). Nanopore sequencing libraries were performed using the Rapid PCR-Barcoding kit (Oxford Nanopore Technologies, Oxford, UK). Samples were multiplexed at 12 samples per flow cell and sequenced for 72 hours.

Taxonomic assignation was performed using the Kraken2 taxonomic sequence classification system (Bayesian Reestimation of Abundance with Kraken, Johns Hopkins University Center for Computational Biology, Baltimore, MD) with a curated database containing publicly available genomes from RefSeq (https://www.ncbi.nlm.nih.gov/refseq/) and metagenomics studies. Estimation of abundance at the species level was done with Bracken (Bayesian Reestimation of Abundance with Kraken, John's Hopkins University Center for Computational Biology, Baltimore, MD). Counts were normalized using Center Log Ratio (CLR) with the R package, zCompositions, to impute zeros.

Ecological diversity analysis was performed using the R packages Phyloseq and Vegan were used to perform alpha diversity analysis. For the alpha diversity analysis, library size differences were adjusted first using the rarefaction method.

Overall characterization of the microbial community at the species level (using raw counts and center-log ratio transformed data) and the derived measures (such as Shannon diversity) were determined for the microbial community. Centered log-ratio (CLR) values of the species of interest (e.g., Actinobacteria, Bacteroidaceae, Bifidobacterium, Collinsella, Enterobacteriaceae, Enterococcus faecalis, Escherichia coli, Faecalibacterium, Peptostreptococcaceae, Prevotella, Roseburia, Ruminococcaceae, Bacteroidetes, Salmonella, Lactobacillus, and Enterococcus hirae), Shannon Diversity values, and standard deviations are summarized in Table 14.

TABLE 12
Effects of Botanical Extracts on the Relative Abundance of Bacterial
Species of Interest in the Feline Microbiome (n = 6)

| | | | | | | Velvet | |
|---|---|---|---|---|---|---|---|
| | | | | | | Foot | |
| | | | | | Dandelion | Winter | Sugar |
| | | Blank | Blank | Inulin | Leaf | Mushroom | Apple |
| | | 0 h | 24 h | 24 h | 24 h | 24 h | 24 h |
| Actinobacteria | Mean | 3.63 | 3.36 | 4.00 | 3.38 | 3.99 | 3.54 |
| | Std | 0.99 | 0.52 | 1.07 | 0.76 | 1.64 | 1.04 |
| Bacteroidaceae | Mean | 8.95 | 10.07 | 10.63 | 9.86 | 10.11 | 10.26 |
| | Std | 0.47 | 0.43 | 0.33 | 0.37 | 0.26 | 0.34 |
| Bifidobacterium | Mean | 2.82 | 2.94 | 4.23 | 3.65 | 4.70 | 2.99 |
| | Std | 4.28 | 3.02 | 3.68 | 2.90 | 3.87 | 2.77 |
| Collinsella | Mean | 6.64 | 6.07 | 6.95 | 6.25 | 6.49 | 6.66 |
| | Std | 0.55 | 0.32 | 0.90 | 0.50 | 0.94 | 0.93 |
| Desulfovibrio | Mean | 0.25 | 0.65 | −1.10 | −1.05 | −1.00 | −0.50 |
| | Std | 2.89 | 3.54 | 2.47 | 3.24 | 2.94 | 3.33 |
| Enterobacteriaceae | Mean | 2.64 | 8.98 | 8.32 | 6.17 | 7.97 | 8.62 |
| | Std | 1.46 | 0.87 | 1.45 | 2.52 | 1.59 | 1.16 |
| Enterococcus_{—}faecalis | Mean | −1.70 | 0.67 | −0.05 | 0.21 | 1.07 | 0.95 |
| | Std | 0.40 | 4.02 | 3.45 | 2.99 | 4.10 | 3.30 |
| Enterococcus_{—}hirae | Mean | −1.70 | −1.60 | 0.32 | −1.70 | 0.20 | 1.10 |
| | Std | 0.40 | 0.34 | 2.52 | 0.37 | 2.64 | 3.31 |
| Escherichia_{—}coli | Mean | 1.69 | 10.56 | 9.87 | 7.76 | 9.53 | 10.05 |
| | Std | 3.84 | 0.86 | 1.46 | 2.46 | 1.67 | 1.20 |
| Eubacterium | Mean | −2.35 | −2.36 | 0.11 | −2.39 | −2.14 | −2.01 |
| | Std | 0.55 | 0.45 | 3.20 | 0.47 | 0.42 | 0.47 |
| Faecalibacterium | Mean | 6.51 | 2.92 | 1.48 | 5.93 | 0.94 | 1.91 |
| | Std | 0.84 | 2.66 | 2.90 | 1.01 | 3.13 | 3.49 |
| Lactobacillus | Mean | −0.74 | −0.82 | −0.66 | −0.66 | −0.32 | 0.27 |
| | Std | 4.31 | 3.89 | 3.54 | 4.19 | 4.59 | 5.04 |
| Peptostreptococcaceae | Mean | 4.16 | 4.39 | 5.16 | 4.44 | 4.32 | 5.42 |
| | Std | 3.61 | 3.77 | 2.62 | 2.21 | 2.45 | 2.73 |
| Prevotella | Mean | 10.09 | 5.23 | 5.36 | 7.46 | 5.71 | 4.99 |
| | Std | 1.68 | 1.01 | 0.89 | 2.19 | 0.78 | 1.05 |
| Roseburia | Mean | 3.02 | 1.43 | 4.18 | 0.99 | 1.22 | 3.12 |
| | Std | 2.65 | 2.63 | 0.66 | 2.33 | 2.81 | 0.74 |
| Ruminococcaceae | Mean | 6.70 | 6.80 | 5.49 | 6.60 | 5.74 | 6.30 |
| | Std | 0.37 | 0.40 | 0.62 | 0.24 | 0.60 | 0.60 |
| Salmonella | Mean | −2.35 | 1.74 | 0.04 | −1.17 | −0.08 | 0.44 |
| | Std | 0.55 | 3.44 | 3.13 | 3.04 | 3.05 | 3.38 |
| Streptococcus | Mean | −1.47 | 1.35 | 1.34 | 0.08 | 0.35 | 2.78 |
| | Std | 1.92 | 2.62 | 3.60 | 2.94 | 4.42 | 3.45 |
| Bacteroidetes | Mean | 7.88 | 7.76 | 8.03 | 7.59 | 7.78 | 7.72 |
| | Std | 0.37 | 0.18 | 0.21 | 0.19 | 0.32 | 0.21 |
| Shannon | Mean | 3.36 | 3.12 | 2.69 | 3.32 | 3.02 | 3.08 |
| | Std | 0.25 | 0.29 | 0.27 | 0.38 | 0.41 | 0.37 |

### Example 7: Effects of Botanical Extracts on Cell Monolayer Integrity

This example measured the transepithelial electrical resistance across a model cell line to evaluate the epithelial barrier integrity at an air-liquid interface in response to various botanical extracts.

The pre-plated human colon adenocarcinoma cell line Caco-2 (i.e., CacoReady cells) were obtained from Adari Cell (Oakland, CA, USA). Upon receipt, shipping media was replaced with complete media consisting of Dulbecco's Minimum Essential Medium (Mediatech Inc, Manassas, VA, USA) supplemented with 1% Penicillin Streptomycin solution and 10% heat-inactivated Fetal Bovine Serum (Life Technologies, Burlington, Canada). Complete media was replaced every other day until Day 21 according to manufacturing instructions. The CacoReady cells were incubated at 37° C. with a humidified atmosphere of 5% CO₂.

The permeability of the cell monolayer was monitored on Day 21 by measuring the transepithelial electrical resistance (TER) in complete culture medium at 37° C., using the REMS autosampler (World Precision Instruments). Only wells that had TER values of 1,000 Ω*cm² or higher were used for the experiment.

Samples for TER analyses were obtained by subjecting various botanical extracts to an in-vitro fermentation model using fecal microbiome from 6 different feline donors. The resulting supernatants from the 6 donors were pooled for each treatment. On day 21 of cell differentiation, baseline (e.g., at time equals 0 hours) TER values were measured. Media was then replaced, and treatments were added at 0.625% (v/v) to the apical chamber and cells were exposed for 24 hours. As a positive control, cells were treated with the anti-inflammatory compound 2-[(aminocarbonyl)amino]-5-(4-fluorophenyl)-3-thiophenecarboxamide (TPCA-1). After the 24 h incubation with test product, the apical chambers of the transwells were treated with an inflammation-stimulating cytokine cocktail of Tumor Necrosis factor alpha (TNF-α) and interleukin-1 beta (IL-1β) for 18 h. After 18 h incubation, TER values were measured for each sample.

The ability of the botanical extracts to reduce the extent of inflammation-induced epithelial permeability on differentiated human intestinal Caco-2 cell monolayer was examined. A decrease in TER value after being exposed to the inflammatory cytokine cocktail represents a reduction in the integrity of the Caco-2 cell monolayer and is a representative model of “leaky gut”. A TER Ratio was calculated by dividing the TER value at 18 h by the baseline TER value at Oh for each individual well. A value below 1 represents a decrease in TER from the initial time 0 such that the lower the value, the more permeable the cell monolayer became after incubation and the closer the value was to 1, the lesser the extent of inflammation-induced permeability and the more protective the treatment. Thus, botanical extracts that reduce or ameliorate the inflammation-induced decrease in TER (e.g., values near 1) are likely to be beneficial for overall gut health.

Table 12 represents a 24-hour pre-treatment of botanical extracts and an additional 18-hour incubation period with or without the addition of the inflammatory stimulus. Treatments show no reduction in TER suggesting that treatments without exposure to the inflammatory stimulus do not cause a significant decrease in TER. No significant decrease in TER was observed for treatments dandelion and velvet foot winter mushroom. The cell monolayer integrity was preserved when exposed to the inflammatory agonist for 18 h. This suggests that the treatments protect cell monolayer integrity and permeability following the exposure of an inflammatory stimulus. TPCA-1 (i.e., 2-[(Aminocarbonyl)amino]-5-(4-fluorophenyl)-3-thiophenecarboxamide), a potent and selective inhibitor of I-kappa-kinase-2, was used as a control, and maintained cell monolayer integrity after exposure to the cytokine cocktail. Baseline TER (0 hours) was measured before the 24-hour pre-treatment of botanical extracts, and after an additional 18-hour with or without an inflammatory stimulus (i.e., TNF-α and IL-1β). Treatments were tested in duplicate. Each value presented in Table 13 is the average TER Ratio±Standard error of the mean (SEM) between two sets of experiments.

TABLE 13
Average TER Ratio With or Without TNF-α and IL-1β

| | TER Ratio: | TER Ratio: |
|---|---|---|
| | No TNF-α and IL-1β | With TNF-α and IL-1β |
| Treatment | (SEM) | (SEM) |
| Media Only | 1.146 (0.007) | 0.858 (0.013) |
| TPCA-1 | 1.033 (0.001) | 1.017 (0.021) |
| Blank 0 h | 1.086 (0.039) | 0.861 (0.019) |
| Blank 24 h | 1.152 (0.011) | 0.886 (0.010) |
| Dandelion extract | 1.048 (0.032) | 1.003 (0.007) |
| Velvet Foot Winter | 1.093 (0.051) | 0.978 (0.028) |
| Mushroom extract | | |
| Sugar Apple Extract | 1.193 (0.055) | 0.908 (0.004) |

The definitions described herein apply to all aspects as described unless otherwise stated.

In this document, the terms “a,” “an,” or “the” are used to include one or more than one unless the context clearly dictates otherwise. The term “or” is used to refer to a nonexclusive “or” unless otherwise indicated. All publications, patents, and patent documents referred to in this document are incorporated by reference herein in their entirety, as though individually incorporated by reference. In the event of inconsistent usages between this document and those documents so incorporated by reference, the usage in the incorporated reference is to be considered supplementary to that of this document; for irreconcilable inconsistencies, the usage in this document controls.

Values expressed in a range format are to be interpreted in a flexible manner to include not only the numerical values explicitly recited as the limits of the range, but also to include all the individual numerical values or sub-ranges encompassed within that range as if each numerical value and sub-range were explicitly recited. For example, a range of “about 0.1% to about 5%” or “about 0.1% to 5%” is to be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

Unless expressly stated, ppm (parts per million), percentage, and ratios are on a by weight basis. Percentage on a by weight basis (% w/w or w/w %) is also referred to as weight percent (wt. %) or percent by weight (% wt.) herein.

### ADDITIONAL EXAMPLES

The following is a non-comprehensive list of additional examples of the invention.
Example 1. A botanical composition comprising: at least one botanical extract; wherein the botanical extract is present in the botanical composition in an amount effective to produce a beneficial effect on a gut microbiome of an animal; wherein the botanical extract comprises one or more botanical extracts comprising an extract of dandelion leaf, dandelion leaf, ground dandelion leaf, dandelion root, Japanese ginseng, asafoetida, privit, common fennel, Indian mulberry, Rangoon creeper, long pepper, Indian lotus, cayenne/chili pepper, velvet foot winter mushroom, sugar apple, lion's mane mushroom, coriander, saw palmetto, Spanish dagger, mound lily yucca, carob, field lupine, goat's head, sweet prickly pear, partridge berry, bugleweed, Stelmatrocryption khasianum, Amaranthus spinosus, and elderberry, or any combinations thereof.
Example 2. The composition of example 1, wherein the botanical extract comprises one or more of an extract of sugar apple, velvet foot winter mushroom, or dandelion leaf.
Example 3. The composition of example 1, wherein a plurality of botanical extracts are present in the botanical composition, each botanical extract present in an amount effective to produce a beneficial effect on the gut microbiome.
Example 4. The composition of any of examples 1-3, wherein the beneficial effect comprises one or more of an increase in a relative abundance of one or more beneficial bacterial strains; a decrease in the relative abundance of one or more pathogenic bacterial strains; an increase the Shannon Diversity index; or an increase in volatile fatty acid concentration produced by the gut microbiome.
Example 5. The composition of example 4, wherein the increase in the relative abundance of the one or more beneficial bacterial strains comprises an increase in a relative abundance of one or more species of Bifidobacterium, Ruminococcaceae, Bacteroidaceae, Faecalibacterium Roseburia, or Prevotella.
Example 6. The composition of example 4, wherein the increase in the relative abundance of the one or more beneficial bacterial strains comprises an increase in the relative abundance of the one or more beneficial bacterial strains in an animal fed a diet containing the botanical composition as compared to an animal fed a diet lacking the botanical composition.
Example 7. The composition of example 4, wherein the increase in the relative abundance of the one or more beneficial bacterial strains comprises an increase in the relative abundance of the one or more beneficial bacterial strains in an animal fed a diet containing the botanical composition compared to a relative abundance of the beneficial strains in an animal experiencing one or more adverse health conditions.
Example 8. The composition of example 4, wherein the decrease in the one or more pathogenic bacterial strains comprises a decrease in the relative abundance of one or more of Burkholderia cepacia, Campylobacter helveticus, Campylobacter jejuni, Clostridium perfringens, Collinsella aerofaciens, Desulfovibrio vulgaris, Enterococcus faecalis, Escherichia coli, Salmonella enterica subsp. enterica serovar Typhimurium, or Streptococcus canis.
Example 9. The composition of example 4, wherein the decrease in the relative abundance of the one or more pathogenic bacterial strains comprises a decrease in the relative abundance of the one or more pathogenic bacterial strains in an animal fed a diet containing the botanical composition relative to a relative abundance of the pathogenic strains that would cause disease.
Example 10. The composition of example 4, wherein the increase in the relative abundance of one or more beneficial bacterial strains comprises an increase in a Bifidobacterium population in an animal fed a diet containing the botanical composition as compared to an animal fed a diet lacking the botanical composition.
Example 11. The composition of example 4, wherein the increase in the relative abundance of one or more beneficial bacterial strains comprises an increase in a Ruminococcaceae population in an animal fed a diet containing the botanical composition as compared to an animal fed a diet lacking the botanical composition.
Example 12. The composition of example 4, wherein the increase in the relative abundance of one or more beneficial bacterial strains comprises an increase in a Bacteroidaceae population in an animal fed a diet containing the botanical composition as compared to an animal fed a diet lacking the botanical composition.
Example 13. The composition of example 4, wherein the increase in the relative abundance of one or more beneficial bacterial strains comprises an increase in a Faecalibacterium population in an animal fed a diet containing the botanical composition as compared to an animal fed a diet lacking the botanical composition.
Example 14. The composition of example 4, wherein the increase in the volatile fatty acid concentration produced by the gut microbiome comprises an increase in the total volatile fatty acid concentration produced by the gut microbiome in an animal fed a diet containing the botanical composition as compared to an animal fed a diet lacking the botanical composition.
Example 15. The composition of example 4, wherein the increase in the volatile fatty acids produced by the gut microbiome comprises an increase in one or more of acetate, propionate, butyrate, and valerate, or derivatives thereof.
Example 16. The composition of example 4, wherein the increase in volatile fatty acid production by the gut microbiome comprises an increase in the acetate concentration produced by the gut microbiome in an animal fed a diet containing the botanical composition as compared to an animal fed a diet lacking the botanical composition.
Example 17. The composition of example 4, wherein the increase in volatile fatty acid production by the gut microbiome comprises an increase of the butyrate concentration produced by the gut microbiome in an animal fed a diet containing the botanical composition as compared to an animal fed a diet lacking the botanical composition.
Example 18. The composition of example 4, wherein the increase in volatile fatty acid production by the gut microbiome comprises an increase in the propionate concentration produced by the gut microbiome in an animal fed a diet containing the botanical composition as compared to an animal fed a diet lacking the botanical composition.
Example 19. The composition of any of examples 1-18, wherein the animal is a feline animal.
Example 20. A method for feeding felines comprising feeding the botanical composition of any one of examples 1-19.
Example 21. The composition of any one of examples 1-20, wherein the amount of botanical extract effective to produce a beneficial effect on a gut health of an animal is from 0.0001 wt. % to 10 wt. %, or from 0.01 wt. % to 5.0 wt. %, or from 0.1 wt. % to 1.0 wt. % of a diet of an animal.
Example 22. A method for modulating a gut microbiome of an animal, comprising: administering to the animal a botanical composition comprising a botanical extract in an amount effective to produce a beneficial effect on a gut health of an animal; wherein the amount of botanical extract effective to produce a beneficial effect causes one or more of: measuring the beneficial effect by determining one or more of: an increase in a relative abundance of one or more beneficial bacterial strains; a decrease in a relative abundance of one or more pathogenic or detrimental bacterial strains; an increase in a Shannon Diversity index; and a change in a concentration of volatile fatty acid produced by the gut microbiome; and wherein the botanical composition comprises one or more botanical extracts comprising an extract of dandelion leaf, dandelion leaf, ground dandelion leaf, dandelion root, Japanese ginseng, asafoetida, privit, common fennel, Indian mulberry, Rangoon creeper, long pepper, Indian lotus, cayenne/chili pepper, velvet foot winter mushroom, sugar apple, lion's mane mushroom, coriander, saw palmetto, Spanish dagger, mound lily yucca, carob, field lupine, goat's head, sweet prickly pear, partridge berry, bugleweed, Stelmatrocryption khasianum, Amaranthus spinosus, and elderberry, or combinations thereof.
Example 23. The method of any example 22, wherein the increase in the relative abundance of the one or more beneficial bacterial strains comprises an increase in a relative abundance of one or more of Bifidobacterium, Ruminococcaceae, Bacteroidaceae, or Faecalibacterium, Lactobacillus, Roseburia, or Prevotella.
Example 24. The method of any one of examples 22-23, wherein the animal is a feline animal.
Example 25. The method of any of examples 22-24, wherein the amount of botanical extract effective to produce a beneficial effect on a gut microbiome of an animal is from 0.0001 wt. % to 10 wt. %, or from 0.1 wt. % to 5.0 wt. %, or from 0.1 wt. % to 1.0 wt. %.
Example 26. The method of example 22, wherein the one or more pathogenic bacterial strains comprise one or more of Burkholderia cepacia, Campylobacter helveticus, Campylobacter jejuni, Clostridium perfringens, Collinsella aerofaciens, Desulfovibrio vulgaris, Enterococcus faecalis, Escherichia coli, Salmonella enterica subsp. enterica serovar Typhimurium, or Streptococcus canis.
Example 27. The method of example 22, wherein measuring the beneficial effect is determined by comparing the effects observed in an animal administered a diet containing the botanical composition as compared to an animal fed a diet lacking the botanical composition.
Example 28. The method of example 22, wherein the increase in the relative abundance of the one or more beneficial bacterial strains comprises an increase in a relative abundance of one or more species of Bifidobacterium, Ruminococcaceae, Bacteroidaceae, or Faecalibacterium.
Example 29. The method of example 22, wherein the increase in the relative abundance of the one or more beneficial bacterial strains comprises an increase in the relative abundance of the one or more beneficial bacterial strains in an animal fed a diet containing the botanical composition as compared to an animal fed a diet lacking the botanical composition.
Example 30. The method of example 22, wherein the increase in the relative abundance of the one or more beneficial bacterial strains comprises an increase in the relative abundance of the one or more beneficial bacterial strains in an animal fed a diet containing the botanical composition compared to a relative abundance of the beneficial strains in an animal experiencing one or more adverse health conditions.
Example 31. The method of example 22, wherein the decrease in the one or more pathogenic bacterial strains comprises a decrease in the relative abundance of one or more of Burkholderia cepacia, Campylobacter helveticus, Campylobacter jejuni, Clostridium perfringens, Collinsella aerofaciens, Desulfovibrio vulgaris, Enterococcus faecalis, Escherichia coli, Salmonella enterica subsp. enterica serovar Typhimurium, or Streptococcus canis.
Example 32. The method of example 22, wherein the decrease in the relative abundance of the one or more pathogenic bacterial strains comprises a decrease in the relative abundance of the one or more pathogenic bacterial strains in an animal fed a diet containing the botanical composition relative to a relative abundance of the pathogenic strains that would cause disease.
Example 33. The method of example 22, wherein the increase in Bifidobacterium comprises an increase in the relative abundance of the Bifidobacterium bacterial population in an animal fed a diet containing the botanical composition as compared to an animal fed a diet lacking the botanical composition.
Example 34. The method of example 21, wherein the increase in Ruminococcaceae comprises an increase in the relative abundance of the Ruminococcaceae bacterial population in an animal fed a diet containing the botanical composition as compared to an animal fed a diet lacking the botanical composition.
Example 35. The method of any of examples 22-34, wherein an increase in Bacteroidaceae comprises an increase in the Bacteroidaceae bacterial population in an animal fed a diet containing the botanical composition as compared to an animal fed a diet lacking the botanical composition.
Example 36. The method of any of examples 22-35 wherein an increase in Faecalibacterium comprises an increase in the relative abundance of the Faecalibacterium bacterial population in an animal fed a diet containing the botanical composition as compared to an animal fed a diet lacking the botanical composition.
Example 37. The method of any of examples 22-36, wherein the change in the volatile fatty acid production by the gut microbiome comprises an increase in a total volatile fatty acid concentration produced by the gut microbiome in an animal fed a diet containing the botanical composition as compared to an animal fed a diet lacking the botanical composition.
Example 38. The method of any of examples 22-37, wherein the change in the concentration of volatile fatty acids by the gut microbiome comprises an increase in one or more of acetate, propionate, isobutyrate, butyrate, isovalerate, and valerate, or derivatives thereof.
Example 39. The method of any of examples 21-38, wherein the change in the concentration of volatile fatty acids is measured in an animal fed a diet containing the botanical composition as compared to an animal fed a diet lacking the botanical composition.
Example 40. The method of any of examples 22-39, wherein the change in the concentration of volatile fatty acids is measured in an animal in a disease state as compared to a baseline concentration of volatile fatty acids in the animal in a non-diseased state.
Example 41. The method of example 40, wherein the disease state comprises diarrhea, obesity, diabetes, inflammatory bowel disease, primary gastrointestinal disease, and severe gastrointestinal disease.
Example 42. The method of any of examples 22-41, further comprising reducing one or more symptoms or intestinal distress in the animal, the symptoms comprising acute diarrhea, chronic diarrhea, constipation, and vomiting.
Example 43. The method of any of examples 22-42, wherein administering to the animal a botanical composition comprising a botanical composition produces at least one health benefit to a feline having one or more health conditions comprising diarrhea, obesity, diabetes, irritable bowel disease, and primary gastrointestinal disease.
Example 44. The method of example 43, wherein at least one health benefit comprises cessation of diarrhea, a decrease in obesity, a reduction in diabetic biomarkers, an improvement of irritable bowel disease, or a decrease in incidence of primary gastrointestinal disease.

1. A botanical composition comprising:
at least one botanical extract;
wherein the botanical extract is present in the botanical composition in an amount effective to produce a beneficial effect on a gut microbiome of an animal;
wherein the botanical extract comprises one or more botanical extracts comprising an extract of dandelion leaf, dandelion leaf, ground dandelion leaf, dandelion root, Japanese ginseng, asafoetida, privit, common fennel, Indian mulberry, Rangoon creeper, long pepper, Indian lotus, cayenne/chili pepper, velvet foot winter mushroom, sugar apple, lion's mane mushroom, coriander, saw palmetto, Spanish dagger, mound lily yucca, carob, field lupine, goat's head, sweet prickly pear, partridge berry, bugleweed, Stelmatrocryption khasianum, Amaranthus spinosus, and elderberry, or any combinations thereof.

2. The composition of claim 1, wherein the botanical extract comprises one or more of an extract of sugar apple, velvet foot winter mushroom, or dandelion leaf.

3. The composition of claim 1, wherein a plurality of botanical extracts are present in the botanical composition, each botanical extract present in an amount effective to produce a beneficial effect on the gut microbiome.

4. The composition of claim 1, wherein the beneficial effect comprises one or more of an increase in a relative abundance of one or more beneficial bacterial strains; a decrease in the relative abundance of one or more pathogenic bacterial strains; an increase the Shannon Diversity index; or an increase in volatile fatty acid concentration produced by the gut microbiome.

5. The composition of claim 4, wherein the increase in the relative abundance of the one or more beneficial bacterial strains comprises an increase in a relative abundance of one or more species of Bifidobacterium, Ruminococcaceae, Bacteroidaceae, Faecalibacterium Roseburia, or Prevotella.

6. The composition of claim 4, wherein the decrease in the one or more pathogenic bacterial strains comprises a decrease in the relative abundance of one or more of Burkholderia cepacia, Campylobacter helveticus, Campylobacter jejuni, Clostridium perfringens, Collinsella aerofaciens, Desulfovibrio vulgaris, Enterococcus faecalis, Escherichia coli, Salmonella enterica subsp. enterica serovar Typhimurium, or Streptococcus canis.

7. The composition of claim 4, wherein the increase in the relative abundance of one or more beneficial bacterial strains comprises an increase in a Bifidobacterium population in an animal fed a diet containing the botanical composition as compared to an animal fed a diet lacking the botanical composition.

8. The composition of claim 4, wherein the increase in the relative abundance of one or more beneficial bacterial strains comprises an increase in a Ruminococcaceae population in an animal fed a diet containing the botanical composition as compared to an animal fed a diet lacking the botanical composition.

9. The composition of claim 4, wherein the increase in the relative abundance of one or more beneficial bacterial strains comprises an increase in a Bacteroidaceae population in an animal fed a diet containing the botanical composition as compared to an animal fed a diet lacking the botanical composition.

10. The composition of claim 4, wherein the increase in the relative abundance of one or more beneficial bacterial strains comprises an increase in a Faecalibacterium population in an animal fed a diet containing the botanical composition as compared to an animal fed a diet lacking the botanical composition.

11. The composition of claim 4, wherein the increase in the volatile fatty acid concentration produced by the gut microbiome comprises an increase in the total volatile fatty acid concentration produced by the gut microbiome in an animal fed a diet containing the botanical composition as compared to an animal fed a diet lacking the botanical composition.

12. The composition of claim 4, wherein the increase in the volatile fatty acids produced by the gut microbiome comprises an increase in the concentration of one or more of acetate, propionate, butyrate, and valerate, or derivatives thereof.

13. A method for feeding felines comprising feeding the botanical composition of claim 1.

14. The composition of claim 1, wherein the amount of botanical extract effective to produce a beneficial effect on a gut health of an animal is from 0.0001 wt. % to 10 wt. %, or from 0.01 wt. % to 5.0 wt. %, or from 0.1 wt. % to 1.0 wt. % of a diet of an animal.

15.-29. (canceled)
